(19) United States
(12) Patent Application Publication
(10) Pub. No.: US 2025/0369022 A1
(43) Pub. Date: Dec. 4, 2025

(54) PRODUCTION OF NATURAL GUM BASE INGREDIENTS BY PLANT CELL FERMENTATION AND APPLICATION THEREOF IN CONFECTIONERY

(71) Applicant: WM. WRIGLEY JR. COMPANY, Chicago, IL (US)

(72) Inventors: Xiaohu XIA, Deerfield, IL (US); Bethany BERNIER, Chicago, IL (US); Michael RUSSELL, Evergreen Park, IL (US)

(73) Assignee: WM. WRIGLEY JR. COMPANY, Chicago, IL (US)

(21) Appl. No.: 19/127,628

(22) PCT Filed: Nov. 6, 2023
(86) PCT No.: PCT/US2023/078766

§ 371 Date: May 6, 2025

Related U.S. Application Data
(60) Provisional application No. 63/423,173, filed on Nov. 7, 2022.

(51) Int. Cl. C12P 5/00; A23G 4/06; A23G 4/08

(52) U.S. Cl. CPC C12P 5/007; A23G 4/064; A23G 4/066; A23G 4/068; A23G 4/08

(57) ABSTRACT

Described herein are cultured gum base ingredients and methods of producing thereof by plant cell culture, also known as plant cell fermentation. The cultured gum base ingredients described herein may comprise one or more terpenoid compounds, such as triterpenoid acetates and polyisoprene. The method of producing the gum base ingredients comprises culturing in a culture medium of a plurality of plant cells derived from a plant that produces a gum base ingredient.

### FIELD

The present disclosure relates generally to food compositions, including gum base compositions and chewing gum compositions, and more specifically to cultured gum base ingredients produced via a plant cell culture process.

### BACKGROUND

The fundamental components of a chewing gum are typically a water-insoluble gum base portion and a water-soluble bulking agent portion. Natural gum base ingredients extracted from chicle, jelutong, and guayule are facing challenges such as supply shortage, sustainability concerns, and material consistency. Such natural gum base ingredients are typically blends of polymers, resins, and inorganic substances, where the polymer polyisoprene drives the elasticity of gum base. There are over 180 plant species that produce natural rubbers. However, with very few exceptions, these natural rubbers and resins currently lack the supply infrastructure needed to source gum base elastomers in the bulk required to meet present market demand, which exceeds 100 megatons (MT) per year. Currently, natural gum base is mainly harvested from the “chicozapote” trees growing in the rainforests manually by Chicleros. Chicozapote trees are not cultivated commercially as the plant requires the supporting biodiversity of the forest for the tree to be productive. This results in strict limitations on the supply of natural gum base from chicozapote and similar trees, as well as environmental concerns regarding forest management as demand for natural gum base increases.

Therefore, there is a need in the art for natural gum base ingredients that are available in sufficient bulk quantities to supply commercial gum production processes for the production of natural chewing gums.

### BRIEF SUMMARY

Disclosed herein are methods and compositions for producing natural gum base ingredients, such as polyisoprene and triterpenoid acetates, through plant cell fermentation, also known as plant cell culture. Plant cell fermentation is a controlled biotechnological process that provides a sustainable and scalable supply solution for plant natural products. Plant cell fermentation provides a viable alternative way to produce natural gum base ingredients in a defined fermentation environment and provides a better sourcing option to the chewing gum industry in terms of sustainability, consistency, cost, and availability at a commercial scale.

In some aspects, provided herein is a method of producing a cultured gum base ingredient, the method comprising culturing in a culture medium a plurality of plant cells derived from a plant that produces the gum base ingredient, thereby producing the cultured gum base ingredient. In some embodiments, the method comprises culturing in a culture medium a plurality of plant cells derived from a plant of a genus selected from the group consisting of Abies, Apios, Brosimum, Castilla, Cnidoscolus, Couma, Dyera, Ficus, Hevea, Isonandra, Lacmellea, Liquidambar, Manilkara, Micropholis, Palaquium, Parthenium, Picea, Pinus, Pistacia, Sideroxylon, and Taraxacum, thereby producing the cultured gum base ingredient. In certain embodiments, the cultured gum base ingredient comprises one or more terpenoid compounds.

In some aspects, provided herein is a method of producing a cultured gum base ingredient comprising one or more terpenoid compounds, the method comprising culturing in a culture medium a plurality of plant cells derived from a plant that produces the gum base ingredient, thereby producing the cultured gum base ingredient. In some embodiments, the plurality of plant cells is derived from a plant of a family selected from the group consisting of Apocynaceae, Asteraceae, Euphorbiaceae, Fabaceae, Hamamelidaceae, Moraceae, Pinaceae, Pistaciaceae, and Sapotaceae. In certain embodiments, the plurality of plant cells is derived from a plant of a genus selected from the group consisting of Abies, Apios, Brosimum, Castilla, Cnidoscolus, Couma, Dyera, Ficus, Hevea, Isonandra, Lacmellea, Liquidambar, Manilkara, Micropholis, Palaquium, Parthenium, Picea, Pinus, Pistacia, Sideroxylon, and Taraxacum.

In some embodiments of the preceding aspects, the plurality of plant cells is derived from a plant of a species selected from the group consisting of Abies amabilis, Abies balsamea, Apios americana, Brosimum utile, Castilla fallax, Cnidoscolus elasticus, Cnidoscolus tepiquensis, Couma macrocarpa, Dyera costulata, Dyera lowii, Ficus platyphylla, Hevea brasiliensis, Isonandra gutta, Lacmellea standleyi, Liquidambar orientalis, Liquidambar styraciflua, Manilkara chicle, Manilkara solimoesensis, Palaquium gutta, Palaquium leiocarpum, Parthenium argentatum, Picea rubens, Picea stichensis, Pinus ponderosa, Pinus lambertiana, Pistacia lentiscus, and Taraxacum kok-saghyz. In some variations, the plurality of plant cells is derived from a plant of a genus selected from the group consisting of Couma, Dyera, Hevea, Manilkara, Parthenium, Pistacia, and Taraxacum. In some embodiments, the plurality of plant cells is derived from a plant of a species selected from the group consisting of Couma macrocarpa, Dyera costulata, Dyera lowii, Hevea brasiliensis, Manilkara chicle, Manilkara solimoesensis, Parthenium argentatum, Pistacia lentiscus, and Taraxacum kok-saghyz. In certain embodiments, the plurality of plant cells is derived from a plant of the genus Manilkara. In certain embodiments, the plurality of plant cells is derived from Manilkara chicle or Manilkara solimoesensis.

In some embodiments of the preceding aspects, which may be combined with any of the preceding embodiments, the one or more terpenoid compounds comprise one or more polyterpenoids, one or more triterpenoid acetates, one or more resin acids, or any combination thereof. In some variations, the one or more terpenoid compounds comprise luperol acetate, beta-amyrin acetate, taraxasterol acetate, alpha-amyrin acetate, polyisoprene, one or more abietic type acids, one or more pimaric type acids, or any combination thereof. In some variations, the cultured gum base ingredient comprises a single terpenoid compound. In other variations, the cultured gum base ingredient comprises a mixture of two or more terpenoid compounds. In some variations, the plurality of plant cells produces at least 0.01 mg/L, at least 1 mg/L, or at least 1 g/L of terpenoid compounds in the culture medium. In certain variations, the plurality of plant cells produces: (i) at least 0.01 mg/L of triterpenoid acetates in the culture medium; (ii) at least 0.01 mg/L of polyterpenoids in the culture medium; and/or (iii) at least 0.01 mg/L of resin acids in the culture medium. In certain variations, the plurality of plant cells produce: (i) at least 0.01 mg/L of luperol acetate in the culture medium; (ii) at least 0.01 mg/L of beta-amyrin acetate in the culture medium; (iii) at least 0.01 mg/L of taraxasterol acetate in the culture medium; (iv) at least 0.01 mg/L of alpha-amyrin acetate in the culture medium; (v) at least 0.01 mg/L of polyisoprene in the culture medium; (vi) at least 0.01 mg/L of one or more abietic type acids; and/or (vii) at least 0.01 mg/L of one or more pimaric type acids.

In some embodiments of the preceding aspects, which may be combined with any of the preceding embodiments, the method further comprises concentrating the culture medium to produce a cultured gum base ingredient concentrate. In some embodiments, which may be combined with any of the preceding embodiments, the method further comprises separating the cultured gum base ingredient from the culture medium or gum base ingredient concentrate to produce an isolated or purified cultured gum base ingredient.

In some aspects, provided herein is a cultured gum base ingredient produced according to any one of the embodiments of the preceding methods.

In some aspects, provided herein is a cultured gum base ingredient concentrate produced according to any one of the embodiments of the preceding methods, comprising terpenoid compounds in an amount of at least 5%, at least 25%, or at least 50% by weight. In some embodiments, the cultured gum base ingredient concentrate comprises: (i) triterpenoid acetates in an amount of at least 5% by weight; (ii) polyterpenoids in an amount of at least 5% by weight; and/or (iii) resin acids in an amount of at least 5% by weight. In certain embodiments, the cultured gum base ingredient concentrate comprises: (i) luperol acetate in an amount of at least 1% by weight; (ii) beta-amyrin acetate in an amount of at least 1% by weight; (iii) taraxasterol acetate in an amount of at least 1% by weight; (iv) alpha-amyrin acetate in an amount of at least 1% by weight; (v) polyisoprene in an amount of at least 1% by weight; (vi) at least 0.01 mg/L of one or more abietic type acids; and/or (vii) at least 0.01 mg/L of one or more pimaric type acids.

In some aspects, provided herein is an isolated or purified cultured gum base ingredient produced according to any one of the embodiments of the preceding methods, comprising terpenoid compounds in an amount of at least 5%, at least 25%, or at least 50% by weight. In some embodiments, the isolated or purified cultured gum base ingredient comprises: (i) triterpenoid acetates in an amount of at least 5% by weight; (ii) polyterpenoids in an amount of at least 5% by weight; and/or (iii) resin acids in an amount of at least 5% by weight. In certain embodiments, the isolated or purified cultured gum base ingredient comprises: (i) luperol acetate in an amount of at least 1% by weight; (ii) beta-amyrin acetate in an amount of at least 1% by weight; (iii) taraxasterol acetate in an amount of at least 1% by weight; (iv) alpha-amyrin acetate in an amount of at least 1% by weight; (v) polyisoprene in an amount of at least 1% by weight; (vi) at least 0.01 mg/L of one or more abietic type acids; and/or (vii) at least 0.01 mg/L of one or more pimaric type acids.

In some aspects, provided herein is a process for producing a food product, the process comprising a step of including any of the cultured gum base ingredients, cultured gum base ingredient concentrates, or isolated or purified gum base ingredients described herein. In certain aspects, provided herein is a food product produced according to said process.

In some aspects, provided herein is a process for producing a gum base, the process comprising a step of including any of the cultured gum base ingredients, cultured gum base ingredient concentrates, or isolated or purified gum base ingredients described herein.

In some aspects, provided herein is a gum base produced according to any of the processes for producing a gum base described herein. In some embodiments, the gum base has a complex viscosity of between 104 and 108 Pa*s at a temperature of between 20° C. and 25° C., and between 102 and 105 Pa*s at a temperature of 60° C. and above, as measured by linear viscoelastic (LVE) rheology. In some embodiments, the gum base comprises the cultured gum base ingredient in an amount of between about 1% and about 25% or between about 5% and about 20% by weight of the gum base. In some embodiments, the gum base further comprises one or more additional components selected from the group consisting of filler component, terpene resin, gum resin, polyvinyl acetate, one or more vegetable fats, one or more emulsifiers, one or more waxes, one or more antioxidants, or any combination thereof. In certain embodiments, the gum base comprises: (i) filler component in an amount of between about 0.01% and about 30% by weight of the gum base; (ii) terpene resin in an amount of between about 5% and about 50% by weight of the gum base; (iii) polyvinyl acetate in an amount of between about 0.01% and about 60% by weight of the gum base; (iv) one or more vegetable fats in an amount of between about 5% and about 30% by weight of the gum base; (v) one or more emulsifiers in an amount of between about 0.01% and about 10% by weight of the gum base; (vi) one or more waxes in an amount of between about 0.01% and about 15% by weight of the gum base; and/or (vii) one or more antioxidants in an amount of between about 0.01% and about 1% by weight of the gum base. In some variations, the filler component is talc, calcium carbonate, or a combination thereof.

In some aspects provided herein is a chewing gum comprising any of the gum bases described herein. In some embodiments, the chewing gum comprises the gum base in an amount of between 5% and 95% or between 20% and 40% by weight of the chewing gum. In certain embodiments, all of the components of the chewing gum are derived from natural sources. In certain embodiments, all of the components of the chewing gum are derived from plant sources. In certain embodiments, the chewing gum is free of artificial and synthetic ingredients. In certain embodiments, the chewing gum is free of ingredients that are derived from petroleum.

### DETAILED DESCRIPTION

The following description is presented to enable a person of ordinary skill in the art to make and use the various embodiments. Descriptions of specific compositions, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the claims.

### Cultured Gum Base Ingredients

In some aspects, described herein are cultured gum base ingredients. As used herein, the term “cultured gum base ingredient” typically refers to a compound or a mix of compounds that are produced in a plant cell culture process and are suitable as an ingredient for gum base. The cultured gum base ingredients described herein are generally hydrophobic, sticky substances that resemble natural gum base ingredients, such as chicle, rubbers, resins (e.g., rosins), and the like. The cultured gum base ingredients described herein typically comprise at least one terpenoid compound but may comprise other hydrophobic compounds and polymers. The cultured gum base ingredients described herein may also comprise components in addition to the terpenoid or other hydrophobic compounds, such as other organic or inorganic substances.

In some embodiments, the cultured gum base ingredient comprises a single compound, e.g., a single terpenoid compound. In other embodiments, the cultured gum base ingredient comprises a mix of one, two, three, four five, six, seven, eight, nine, or ten or more compounds, e.g., one, two, three, four five, six, seven, eight, nine, or ten or more terpenoid compounds. In certain embodiments, the cultured gum base ingredient comprises one or more hydrophobic polymers, e.g., one or more hydrocarbon polymers. In certain embodiments, the cultured gum base ingredient comprises one or more inorganic compounds.

In some embodiments, the terpenoid compound is a compound comprising at least one isoprene unit. Terpenoid compounds may include, for example, hemiterpenoids, monoterpenoids, sequiterpenoids, diterpenoids, triterpenoids (e.g., triterpenoid acetates), tetraterpenoids, and polyterpenoids (e.g., polyisoprene). Terpenoid compounds include both modified and unmodified terpenoids. Unmodified terpenes are that consist only of carbon and hydrogen molecules derived from one or more isoprene units, such as limonene (an unmodified monoterpene) or squalene (an unmodified triterpene). Modified terpenoids are compounds that are derived from terpenes and have been modified, for example, to add one or more functional groups, such as in the case of geosmin (a modified sesquiterpenoid) and luperol acetate (a modified triterpenoid).

In some embodiments, the cultured gum base ingredient comprises one or more terpenoid compounds. The cultured gum base ingredients described herein may comprise one or more terpenoid compound known in the art. For example, in some embodiments, the cultured gum base ingredient comprises one or more hemiterpenoids, one or more monoterpenoids, one or more sequiterpenoids, one or more diterpenoids, one or more triterpenoids, one or more tetraterpenoids, and/or one or more polyterpenoids. In some variations, the cultured gum base ingredient comprises one or more terpenoids having a single isoprene unit, one or more terpenoids having two isoprene units, one or more terpenoids having three isoprene units, one or more terpenoids having four isoprene units, one or more terpenoids having six isoprene units (e.g., triterpenoid acetates), one or more terpenoids having eight isoprene units, and/or one or more terpenoids having more than eight isoprene units (e.g., polyisoprene).

In certain embodiments, the cultured gum base ingredient comprises one or more terpenoid compounds comprising one or more polyterpenoids, one or more triterpenoid acetates, one or more resin acids, or any combination thereof. In some embodiments, the cultured gum base ingredient comprises a single terpenoid compound. In other embodiments, the cultured gum base ingredient comprises a mix of one, two, three, four five, six, seven, eight, nine, or ten or more terpenoid compounds. In certain embodiments, the cultured gum base ingredient comprises one or more compounds that are not terpenoid compounds.

In some embodiments, the cultured gum base ingredient comprises one or more terpenoid compounds found in chicle. As used herein, the term “chicle” generally refers to the milky latex secreted by trees of the genus Manilkara that is used as a natural gum base. Chicle has been found to be composed of about 44% luperol acetate, about 16% beta-amyrin acetate, about 6% taraxasterol acetate, about 7% alpha-amyrin acetate, about 16% polyisoprene, and about 11% inorganic substances by dry weight (Reyes-Gómez, Montiel, and Tlenkopatchev. “Chicle gum from sapodilla (Manilkara zapota) as a renewable resource for metathesis transformations.” Journal of the Mexican Chemical Society 62.1 (2018)). In some embodiments, the cultured gum base ingredient is similar to that of chicle such that the cultured gum base ingredient may be used in the same manner as chicle for production of a gum base and, optionally, a chewing gum. Therefore, in some embodiments, the cultured gum base ingredient comprises one, two, three, four, five, or all six of luperol acetate, beta-amyrin acetate, taraxasterol acetate, alpha-amyrin acetate, and polyisoprene. In some embodiments, the cultured gum base ingredient comprises one, two, three, four, five, or all six of luperol acetate in an amount of between 30% and 60% by dry weight; beta-amyrin acetate in an amount of between 1% and 30% by dry weight; taraxasterol acetate in an amount of between 0.1% and 20% by dry weight; alpha-amyrin acetate in an amount of between 0.1% and 25% by dry weight; and polyisoprene in an amount of between 1% and 30% by dry weight. In certain embodiments, the cultured gum base ingredient comprises one, two, three, four, five, or all six of luperol acetate in an amount of between 40% and 50% by dry weight; beta-amyrin acetate in an amount of between 10% and 20% by dry weight; taraxasterol acetate in an amount of between 1% and 11% by dry weight; alpha-amyrin acetate in an amount of between 2% and 12% by dry weight; and polyisoprene in an amount of between 11% and 21% by dry weight. In certain embodiments, the cultured gum base ingredient comprises luperol acetate, beta-amyrin acetate, taraxasterol acetate, alpha-amyrin acetate, and polyisoprene in a ratio of about 9:3:1:1:3 by dry weight.

In some embodiments, the cultured gum base ingredient comprises one or more compounds found in rosins. As used herein, the term “rosin” typically refers to a naturally occurring resin derived from pine and spruce trees that primarily consists of a complex mixture of resin acids and may also include fatty acids (e.g., palmitic acid, oleic acid, and linoleic acid), fatty alcohols, sterols (e.g., unsaponifiable sterols), and/or other alkyl hydrocarbon derivatives. Rosins are also known as resin acids or colophony and include, for example, gum rosin, wood rosin, and tall oil rosin, and are important additives to rubber to tune its rheological properties. Resin acids may include, for example, abietic type acids and pimaric type acids. Abietic type acids may include, for example, abietic acid, neoabietic acid, dehydroabietic acid, palustric acid, and levopimaric acid. Pimaric type acids may include, for example, pimaric acid and isopimaric acid.

The cultured gum base ingredients described herein can comprise one or more terpenoid compounds that are known in the art. Terpenoid compounds may include, for example, abietane, adonirubin, adonixanthin, alloaromadendrene, allyl hexanoate, antheraxanthin, astaxanthin, benzaldehyde, (Z)-α-cis-bergamotene, (Z)-α-trans-bergamotene, β-bisabolol, epi-α-bisabolol, β-bisabolene, borneol (camphol), cis-γ-bisabolene, borneol acetate (bornyl acetate), α-cadinene, camphene, camphor, canthaxanthin, capsorubrin, α-carotene, β-carotene, β,ψ-carotene, δ-carotene, ε-carotene, γ-carotene, 4-keto-γ-carotene, ζ-carotene, cis-carveol, caryophyllene (β-caryophyllene), α-humulene (α-caryophyllene), γ-cadinene, Δ-3-carene, caryophyllene oxide, cembrene A, 1,8-cineole, citral A, citral B, citronellal, citronellol, cinnameldehyde, α-copaene (aglaiene), α-cryptoxanthin, β-cryptoxanthin, γ-curcumene, β-cymene, deoxyflexixanthin, diatoxanthin, dictyophorine A, dictyophorine B, 7,8-didehydroastaxanthin, didehydrolycopene, echinenone, 3-hydroxyechinenone, 3′-hydroxyechinenone, β-elemene, γ-elemene, ethyl decdienoate, ethyl maltol, ethyl propionate, ethylvanillin, eucalyptol, α-eudesmol, β-eudesmol, γ-eudesmol, eugenol, cis-β-farnesene ((Z)-β-farnesene), trans-α-farnesene, trans-β-farnesene, trans-y bisabolene, farnesol, fenchone, fenchol (norbornanol, β-fenchol), fucoxanthin, fucoxanthinol, geraniol, α-guaiene, guaiol, guaiazulene, halomon, hopane, methyl anthranilate, methyl salicylate, 2-methyl-4-heptanone, 3-methyl-4-heptanone, hexyl acetate, ipsdienol, isoamyl acetate, isorenieratene, β-isorenieratene, labdane, lactucaxanthin, lanostane, lemenol, limonene, d-limonene (limonene), linolool (linalyl alcohol, β-linolool), longifolene, α-longipinene, lutein, lycopene, malabaricane, menthol, γ-muurolene, myrcene (β-myrcene), myxobactone, neoxanthin, nerolidol, trans-nerolidol, nerol, neurosporene, hydroxyneurosporene, β-ocimene (cis-ocimene), octyl acetate, oleanane, patchoulol, peridinin, α-phellandrene, phytane, phytoene, phytol, α-pinene (2-pinene), β-pinene, polypodatetraene, pulegone, rhodopin, rhodopin glucoside, 4-keto-rubixanthin, sabinene, cis-sabinene hydrate (cis-thujanol), sclarene, β-selinene, α-selinene, siphonaxanthin, spheroidene, spheroidenone, spirilloxanthin, squalene, stemarene, stemodene, taxadiene, taxane, γ-terpinene, terpinolene (isoterpine), terpineol (α terpineol), terpineol-4-ol, α-terpinene (terpilene), α-thujene (origanene), torulene, 4-keto-torulene, 3-hydroxy-4-keto-torulene, uriolide, uriolide acetate, vanillin, vetivazulene, viridiflorene (ledene), violaxanthin, α-ylangene, zeaxanthin-β-diglucoside, zeaxanthin, and zingiberene.

In some embodiments, the cultured gum base ingredient comprises high molecular wight polymers, such as high molecular weight polyterpenoids. In some variations, the cultured gum base ingredient comprises compounds have an average molecular weight of at least 100 Daltons (Da) or at least 100 g/mol. In some embodiments, the cultured gum base ingredient comprises terpenoid compounds (e.g., polyterpenoids, pentacyclic triterpenoids such as triterpenoid acetates, diterpenoids such as resin acids, sesquiterpenoids, monoterpenoids, or any combination thereof) having an average molecular weight of between about 100 and about 100,000 Da. In certain embodiments, the cultured gum base ingredient comprises a polymer (e.g., a polyterpenoid, e.g., polyisoprene) having an average molecular weight of between about 10,000 Da and about 3,000,000 Da. Methods for determining the molecular weight of polymers are known in the art and may include, for example, gel permeation chromatography (GPC), size exclusion chromatography, light scattering, viscometry, and osmometry.

The cultured gum base ingredient described herein may have certain thermophysical and rheological properties that are suitable for use in production of a gum base. For example, in certain embodiments, the cultured gum base ingredient has a glass transition temperature (Tg) substantially below mouth temperature, e.g., a Tg below a 10° C., and optionally below −76° C. The glass transition temperature of the cultured gum base ingredient can be conventionally measured using Differential Scanning calorimetry (DSC) as is well known in the art. Additionally, in some embodiments, the cultured gum base ingredient has a Tg sufficiently low as to allow convenient and efficient processing by allowing lower processing temperatures and shorter mixing times during gum base production. Further, in some embodiments, the cultured gum base ingredient has a degradation resistance so that it has a thermal degradation (temperature at which 1% of the sample was lost during thermal gravimetric analysis) above 275° C.

### Cultured Gum Base Ingredient Concentrates

In some aspects, provided herein is a cultured gum base ingredient concentrate. In some embodiments, the cultured gum base ingredient concentrate is a composition derived from plant cell culture medium that comprises the cultured gum base ingredient and has been concentrated, e.g., by removing water, to provide a composition that comprises the cultured gum base ingredient at an increased concentration than in the unconcentrated cultured medium. In some embodiments, the cultured gum base ingredient concentrate comprises the cultured gum base ingredient in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In some embodiments, the cultured gum base ingredient concentrate comprises terpenoid compounds in an amount of at least 1% (e.g., at least 1%, at least 5%, at least 10%, at least 15%, at least 20%, at least 25%, at least 30%, at least 40%, at least 50%, at least 60%, at least 70%) by weight or by volume.

In some embodiments which may be combined with the preceding embodiments, the cultured gum base ingredient concentrate comprises triterpenoid acetates in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In certain embodiments which may be combined with the preceding embodiments, the cultured gum base ingredient concentrate comprises luperol acetate in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In certain embodiments which may be combined with the preceding embodiments, the cultured gum base ingredient concentrate comprises beta-amyrin acetate in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In certain embodiments which may be combined with the preceding embodiments, the cultured gum base ingredient concentrate comprises alpha-amyrin acetate in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In some embodiments which may be combined with the preceding embodiments, the cultured gum base ingredient concentrate comprises taraxasterol acetate in an amount of at least 1% (e.g., at least 1%, at least 5%, at least 10%, at least 15%, at least 20%, at least 25%, at least 30%, at least 40%, at least 50%, at least 60%, or at least 70%) by weight or by volume.

In some embodiments which may be combined with the preceding embodiments, the cultured gum base ingredient concentrate comprises polyterpenoids in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In certain embodiments which may be combined with the preceding embodiments, the cultured gum base ingredient concentrate comprises polyisoprene in an amount of at least 1% (e.g., at least 1%, at least 5%, at least 10%, at least 15%, at least 20%, at least 25%, at least 30%, at least 40%, at least 50%, at least 60%, or at least 70%) by weight or by volume.

In some embodiments which may be combined with the preceding embodiments, the cultured gum base ingredient concentrate comprises resin acids in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In certain embodiments which may be combined with the preceding embodiments, the cultured gum base ingredient concentrate comprises one or more abietic type acids in an amount of at least 1% (e.g., at least 1%, at least 5%, at least 10%, at least 15%, at least 20%, at least 25%, at least 30%, at least 40%, at least 50%, at least 60%, or at least 70%) by weight or by volume. In certain embodiments which may be combined with the preceding embodiments, the cultured gum base ingredient concentrate comprises one or more pimaric type acids in an amount of at least 1% (e.g., at least 1%, at least 5%, at least 10%, at least 15%, at least 20%, at least 25%, at least 30%, at least 40%, at least 50%, at least 60%, or at least 70%) by weight or by volume.

In some embodiments, the cultured gum base ingredient concentrate comprises one, two, three, four, five, or all six of luperol acetate, beta-amyrin acetate, taraxasterol acetate, alpha-amyrin acetate, and polyisoprene. In some embodiments, the cultured gum base ingredient concentrate comprises one, two, three, four, five, or all six of luperol acetate in an amount of between 30% and 60% by dry weight; beta-amyrin acetate in an amount of between 1% and 30% by dry weight; taraxasterol acetate in an amount of between 0.1% and 20% by dry weight; alpha-amyrin acetate in an amount of between 0.1% and 25% by dry weight; and polyisoprene in an amount of between 1% and 30% by dry weight. In certain embodiments, the cultured gum base ingredient concentrate comprises one, two, three, four, five, or all six of luperol acetate in an amount of between 40% and 50% by dry weight; beta-amyrin acetate in an amount of between 10% and 20% by dry weight; taraxasterol acetate in an amount of between 1% and 11% by dry weight; alpha-amyrin acetate in an amount of between 2% and 12% by dry weight; and polyisoprene in an amount of between 11% and 21% by dry weight. In certain embodiments, the cultured gum base ingredient concentrate comprises luperol acetate, beta-amyrin acetate, taraxasterol acetate, alpha-amyrin acetate, and polyisoprene in a ratio of about 9:3:1:1:3 by dry weight.

In some embodiments, the cultured gum base ingredient concentrate comprises high molecular wight polymers, such as high molecular weight polyterpenoids. In some embodiments, the cultured gum base ingredient concentrate comprises terpenoid compounds (e.g., polyterpenoids, pentacyclic triterpenoids such as triterpenoid acetates, diterpenoids such as resin acids, sesquiterpenoids, monoterpenoids, or any combination thereof) having an average molecular weight of between about 100 and about 100,000 Da. In certain embodiments, the cultured gum base ingredient concentrate comprises a polymer (e.g., a polyterpenoid, e.g., polyisoprene) having an average molecular weight of between about 10,000 Da and about 3,000,000 Da. Methods for determining the molecular weight of polymers are known in the art and may include, for example, gel permeation chromatography (GPC), size exclusion chromatography, light scattering, viscometry, and osmometry.

The cultured gum base ingredient concentrates described herein may have certain thermophysical and rheological properties that are suitable for use in production of a gum base. For example, in some embodiments, the cultured gum base ingredient concentrate has a glass transition temperature (Tg) substantially below mouth temperature, e.g., a Tg below a 10° C., and optionally below −76° C. The glass transition temperature of the cultured gum base ingredient concentrate can be conventionally measured using Differential Scanning calorimetry (DSC) as is well known in the art. Additionally, in some embodiments, the cultured gum base ingredient concentrate has a Tg sufficiently low as to allow convenient and efficient processing by allowing lower processing temperatures and shorter mixing times during gum base production. Further, in some embodiments, the cultured gum base ingredient concentrate has a degradation resistance so that it has a thermal degradation (temperature at which 1% of the sample was lost during thermal gravimetric analysis) above 275° C.

### Isolated or Purified Cultured Gum Base Ingredient

In some aspects, provided herein is an isolated or purified cultured gum base ingredient. In some embodiments, the isolated or purified cultured gum base ingredient comprises a gum base ingredient derived from a plant cell culture medium at a purity of about 70% or greater. In some embodiments, the isolated or purified cultured gum base ingredient comprises the cultured gum base ingredient in an amount of at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) by weight or by volume. In some embodiments, the isolated or purified cultured gum base ingredient comprises terpenoid compounds in an amount of at least 1% (e.g., at least 1%, at least 5%, at least 10%, at least 15%, at least 20%, at least 25%, at least 30%, at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, or at least 99%) by weight or by volume.

In some embodiments which may be combined with the preceding embodiments, the isolated or purified cultured gum base ingredient comprises triterpenoid acetates in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume. In certain embodiments which may be combined with the preceding embodiments, the isolated or purified cultured gum base ingredient comprises luperol acetate in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume. In certain embodiments which may be combined with the preceding embodiments, the isolated or purified cultured gum base ingredient comprises beta-amyrin acetate in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume. In certain embodiments which may be combined with the preceding embodiments, the isolated or purified cultured gum base ingredient comprises alpha-amyrin acetate in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume. In some embodiments which may be combined with the preceding embodiments, the isolated or purified cultured gum base ingredient comprises taraxasterol acetate in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume.

In some embodiments which may be combined with the preceding embodiments, the isolated or purified cultured gum base ingredient comprises polyterpenoids in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume. In certain embodiments which may be combined with the preceding embodiments, the isolated or purified cultured gum base ingredient comprises polyisoprene in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume.

In some embodiments which may be combined with the preceding embodiments, the isolated or purified cultured gum base ingredient comprises resin acids in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume. In certain embodiments which may be combined with the preceding embodiments, the isolated or purified cultured gum base ingredient comprises one or more abietic type acids in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume. In certain embodiments which may be combined with the preceding embodiments, the isolated or purified cultured gum base ingredient comprises one or more pimaric type acids in an amount of at least 1% (e.g., at least 1%, at least 5%, at least 10%, at least 15%, at least 20%, at least 25%, at least 30%, at least 40%, at least 50%, at least 60%, at least 70%, at least 80%, at least 90%, or at least 95%) by weight or by volume.

In some embodiments, the isolated or purified cultured gum base ingredient comprises one, two, three, four, five, or all six of luperol acetate, beta-amyrin acetate, taraxasterol acetate, alpha-amyrin acetate, and polyisoprene. In some embodiments, the isolated or purified cultured gum base ingredient comprises one, two, three, four, five, or all six of luperol acetate in an amount of between 30% and 60% by dry weight; beta-amyrin acetate in an amount of between 1% and 30% by dry weight; taraxasterol acetate in an amount of between 0.1% and 20% by dry weight; alpha-amyrin acetate in an amount of between 0.1% and 25% by dry weight; and polyisoprene in an amount of between 1% and 30% by dry weight. In certain embodiments, the isolated or purified cultured gum base ingredient comprises one, two, three, four, five, or all six of luperol acetate in an amount of between 40% and 50% by dry weight; beta-amyrin acetate in an amount of between 10% and 20% by dry weight; taraxasterol acetate in an amount of between 1% and 11% by dry weight; alpha-amyrin acetate in an amount of between 2% and 12% by dry weight; and polyisoprene in an amount of between 11% and 21% by dry weight. In certain embodiments, the isolated or purified cultured gum base ingredient comprises luperol acetate, beta-amyrin acetate, taraxasterol acetate, alpha-amyrin acetate, and polyisoprene in a ratio of about 9:3:1:1:3 by dry weight.

In some embodiments, the isolated or purified cultured gum base ingredient comprises high molecular wight polymers, such as high molecular weight polyterpenoids. In some embodiments, the isolated or purified cultured gum base ingredient comprises terpenoid compounds (e.g., polyterpenoids, pentacyclic triterpenoids such as triterpenoid acetates, diterpenoids such as resin acids, sesquiterpenoids, monoterpenoids, or any combination thereof) having an average molecular weight of between about 100 and about 100,000 Da. In certain embodiments, the isolated or purified cultured gum base ingredient comprises a polymer (e.g., a polyterpenoid, e.g., polyisoprene) having an average molecular weight of between about 10,000 Da and about 3,000,000 Da. Methods for determining the molecular weight of polymers are known in the art and may include, for example, gel permeation chromatography (GPC), size exclusion chromatography, light scattering, viscometry, and osmometry.

The isolated or purified cultured gum base ingredients described herein may have certain thermophysical and rheological properties that are suitable for use in production of a gum base. For example, in some embodiments, the isolated or purified cultured gum base ingredient has a glass transition temperature (Tg) substantially below mouth temperature, e.g., a Tg below a 10° C., and optionally below −76° C. The glass transition temperature of the isolated or purified cultured gum base ingredient can be conventionally measured using Differential Scanning calorimetry (DSC) as is well known in the art. Additionally, in some embodiments, the isolated or purified cultured gum base ingredient has a Tg sufficiently low as to allow convenient and efficient processing by allowing lower processing temperatures and shorter mixing times during gum base production. Further, in some embodiments, the isolated or purified cultured gum base ingredient has a degradation resistance so that it has a thermal degradation (temperature at which 1% of the sample was lost during thermal gravimetric analysis) above 275° C.

### Methods of Producing Cultured Gum Base Ingredients

In some aspects, provided herein is a method of producing a cultured gum base ingredient, the method comprising culturing in a culture medium a plurality of plant cells that produce the gum base ingredient. Plant cell culture, also known as plant cell fermentation, provides a means to produce plant natural products on a large scale in a controlled environment, and provides an alternative to harvesting from cultivated or wild plants, which may pose logistical and environmental problems. The methods described herein utilize plant cell culture to produce plant polymers (e.g., rubbers, resins (e.g., rosins), or latexes, or components thereof) suitable for use in producing gum bases.

In some embodiments, the method of producing a cultured gum base ingredient of the present disclosure comprises culturing a plurality of plant cells in a culture medium. The culture medium may be any solid, liquid, or gel in which a plant cell is cultured. Typically, the culture medium comprises all of the nutrients required for the plant cells to grow and produce the cultured gum base ingredient, allowing for the accumulation of the cultured gum base ingredient in the culture medium.

### Plant Species

In some embodiments, the method of producing a cultured gum base ingredient comprises culturing in a culture medium a plurality of plant cells that are derived from a plant that naturally produces a polymer or mixture of polymers suitable for use in producing a gum base. For example, in some embodiments, the method of producing a cultured gum base ingredient comprises culturing in a culture medium a plurality of plant cells that are derived from a plant that naturally produces a chicle, rubber, resin (e.g., rosin), latex, or other substance comprising polymers suitable for use in producing a gum base. In certain embodiments, the method of producing a cultured gum base ingredient comprises culturing in a culture medium a plurality of plant cells that are derived from a plant that naturally produces chicle. The plurality of plant cells can be derived from any plant known in the art, or described herein, known to produce a natural rubber, resin (e.g., rosin), latex, or other substance suitable for use in making a gum base.

In some embodiments, the method of producing a cultured gum base ingredient comprises culturing in a culture medium a plurality of plant cells that are derived from a plant of the family Apocynaceae, Asteraceae, Euphorbiaceae, Fabaceae, Hamamelidaceae, Moraceae, Pinaceae, Pistaciaceae, or Sapotaceae. In some embodiments, the method of producing a cultured gum base ingredient comprises culturing in a culture medium a plurality of plant cells that are derived from a plant of the genus Abies, Apios, Brosimum, Castilla, Cnidoscolus, Couma, Dyera, Ficus, Hevea, Isonandra, Lacmellea, Liquidambar, Manilkara, Micropholis, Palaquium, Parthenium, Picea, Pinus, Pistacia, Sideroxylon, or Taraxacum.

In some embodiments, the method of producing a cultured gum base ingredient comprises culturing in a culture medium a plurality of plant cells that are derived from a plant of the Pinaceae family. In certain embodiments, the plurality of plant cells is derived from a fir, such as a plant from the genus Abies or Pseudotsuga. Firs include, for example, Pacific silver fir (Abies amabilis), Douglas fir (Pseudotsuga menziesii), balsam fir (Abies balsamea), grand fir (Abies grandis), and red fir (Abies magnifica). In certain embodiments, the plurality of plant cells is derived from a pine, such as a plant from the genus Pinus. Pines include, for example, Ponderosa pine (Pinus ponderosa) and sugar pine (Pinus lambertiana). In certain embodiments, the plurality of plant cells is derived from a spruce, such as a plant from the genus Picea. Spruces include, for example, Sitka spruce (Picea sitchensis) and red spruce (Picea rubens).

In some embodiments, the method of producing a cultured gum base ingredient comprises culturing in a culture medium a plurality of plant cells that are derived from a plant of the Euphorbiaceae family, also known as the spurge family. In certain embodiments, the plurality of plant cells is derived from a rubber plant, such as a plant from the genus Hevea. Rubber plants include, for example, the Pará rubber tree (Hevea brasiliensis). In certain embodiments, the plurality of plant cells is derived from a chilte plant, also known as Jatropha, such as a plant from the genus Cnidoscolus or Jatropha. Chilte plants include, for example, Cnidoscolus elasticus and Cnidoscolus tepiquensis.

In some embodiments, the method of producing a cultured gum base ingredient comprises culturing in a culture medium a plurality of plant cells that are derived from a plant of the Apocynaceae family, also known as the dogbane family. In certain embodiments, the plurality of plant cells is derived from a sorva plant, also known as leche caspi, pendare, and perillo, such as a plant from the genus Couma. Sorva plants include, for example, Couma macrocarpa. In certain embodiments, the plurality of plant cells is derived from a jelutong plant, such as a plant from the genus Dyera. Jelutong plants include, for example, Dyera costulata and Dyera lowii.

In some embodiments, the method of producing a cultured gum base ingredient comprises culturing in a culture medium a plurality of plant cells that are derived from a plant of the Sapotaceae family. In certain embodiments, the plurality of plant cells is derived from a chicle plant, also known as Venezuelan chicle, chiquibul, and crown gum, such as a plant from the genus Manilkara. Chicle plants include, for example, chicle tree (Manilkara chicle), chicozapote (Manilkara zapota, also known as sapodilla), massaranduba (Manilkara bidentata), nispero (Manilkara huberi), and Manilkara solimoesensis. In certain embodiments, the plurality of plant cells is derived from a gutta percha plant, such as a plant from the genus Palaquim or Isonandra. Gutta percha plants include, for example, Palaquium gutta, also known as Isonandra gutta. In certain embodiments, the plurality of plant cells is derived from a gutta hang kang plant, such as a plant of the genus Palaquim. Gutta hang kang plants include, for example, Palaquium leiocarpum. In certain embodiments, the plurality of plant cells is derived from a rosidinha plant, also known as rosadinha, such as a plant from the genus Micropholis, also known as Sideroxylon.

In some embodiments, the method of producing a cultured gum base ingredient comprises culturing in a culture medium a plurality of plant cells that are derived from a plant of the Pistaciaceae family. In certain embodiments, the plurality of plant cells is derived from a mastic plant, also known as pistachier lentisque, such as a plant from the genus Pistacia. Mastic plants include, for example, Pistacia lentiscus.

In some embodiments, the method of producing a cultured gum base ingredient comprises culturing in a culture medium a plurality of plant cells that are derived from a plant of the Asteraceae family, also known as the Compositae family. In certain embodiments, the plurality of plant cells is derived from a dandelion plant, such as a plant from the genus Taraxacum. Dandelion plants include, for example, Russian dandelion (Taraxacum kok-saghyz), also known as rubber dandelion. In certain embodiments, the plurality of plant cells is derived from a guayale plant, such as a plant of the genus Parthenium. Guayale plants include, for example, Parthenium argentatum.

In some embodiments, the method of producing a cultured gum base ingredient comprises culturing in a culture medium a plurality of plant cells that are derived from a plant of the Fabaceae family. In certain embodiments, the plurality of plant cells is derived from a groundnut plant, such as a plant of the genus Apios. Ground nut plants include, for example, American groundnut (Apios americana).

In some embodiments, the method of producing a cultured gum base ingredient comprises culturing in a culture medium a plurality of plant cells that are derived from a plant of the Moraceae family, also known as the fig family. In certain embodiments, the plurality of plant cells is derived from a leche de vaca plant, such as a plant of the genus Brosimum or Lacmellea. Leche de vaca plants include, for example, galactodendron (Brosimum utile) and Lacmellea standleyi. In certain embodiments, the plurality of plant cells is derived from a Niger gutta plant, such as a plant from the genus Ficus. Niger gutta plants include, for example, Ficus platyphylla. In certain embodiments, the plurality of plant cells is derived from a tunu plant, also known as tuno, such as a plant from the genus Castilla. Tunu plants include, for example, Castilla fallax.

In some embodiments, the method of producing a cultured gum base ingredient comprises culturing in a culture medium a plurality of plant cells that are derived from a plant of the Hamamelidaceae family, also known as the witch-hazel family. In certain embodiments, the plurality of plant cells is derived from a sweet gum plant, such as a plant from the genus Liquidambar. Sweet gum plants include, for example, American sweetgum (Liquidambar styraciflua, also known as red gum) and Turkish sweetgum (Liquidambar orientalis, also known as oriental sweetgum).

### Plant Cell Line Development

The methods of producing a cultured gum base ingredient described herein comprise culturing a plurality of cells derived from a plant in a culture medium, thereby producing the cultured gum base ingredient. Methods for developing a line of plant cells suitable for plant cell culture known in the art and described in, e.g., Constabel (“Principles underlying the use of plant cell fermentation for secondary metabolite production.” Biochemistry and Cell Biology 66.6 (1988): 658-664); Dunwell (Plant Cell Culture—Present and Future, Plant Cell Culture: Essential Methods, 2010); Hall (“Plant cell culture initiation.” Molecular biotechnology 16.2 (2000): 161-173); Hellwig et al. (“Plant cell cultures for the production of recombinant proteins.” Nature biotechnology 22.11 (2004): 1415-1422); Wilson and Roberts (“Recent advances towards development and commercialization of plant cell culture processes for the synthesis of biomolecules.” Plant biotechnology journal 10.3 (2012): 249-268); and Xu and Zhang (“On the way to commercializing plant cell culture platform for biopharmaceuticals: present status and prospect.” Pharmaceutical bioprocessing 2.6 (2014): 499).

In an exemplary plant cell line development method, one or more source plants may be selected based on availability, genetic diversity, and the ability of the whole plants to generate a desired cultured gum base ingredient. Plant cell lines may be developed from any plant species known in the art or described herein that naturally produce a chicle, rubber, resin (e.g., rosin), or other type of latex suitable as a gum base ingredient.

One method of plant cell line development comprises generating explants from a selected plant, generating calli of de-differentiated cells, and establishing the cells in a plant cell suspension culture. Explants (i.e., plant tissue samples) from a whole plant of the desired species may be obtained from any organ or tissue of the whole plant. Explants may be obtained from the shoot of the plant (e.g., the above-ground portion of the plant comprising the stem and leaves) or from the roots of the plant. Organs and tissues from which explants may be obtain include, for example, a bud (e.g., an apical bud and/or a lateral bud), a leaf or leaf tissue, a stem (e.g., a tree trunk) or stem tissue (e.g., tree bark), or a root or root tissue. Leaf tissues may include, e.g., a leaf primordium, a petiole, a leaf blade, leaf dermal tissue (e.g., cuticle and/or epidermis), leaf ground tissue (e.g., palisade mesophyll and/or spongy mesophyll), and/or leaf vascular tissue (e.g., xylem and/or phloem). Stem tissues may include, e.g., a node, an internode, bark or bark tissues, stem dermal tissue (e.g., epidermis), stem ground tissue (e.g., cortex and/or pith), stem vascular tissue (e.g., xylem and/or phloem), and/or stem meristematic tissue (e.g., vascular cambium). Bark tissues may include, e.g., secondary phloem and/or periderm (e.g., cork, cork cambium, and/or phelloderm). Explants from stem and bark tissues may be obtained from the main stem of a plant (e.g., the main trunk a tree) or from branched stems (e.g., branches of a tree). Root tissues may include, e.g., a taproot, a lateral root, a root apex, a root cap, root dermal tissue (e.g., root hairs and/or epidermis), root ground tissue (e.g., cortex, endodermis, and/or pericycle), root vascular tissue (e.g., xylem and/or phloem), and/or root meristematic tissue (e.g., vascular cambium).

Once explants are obtained, they are optionally sterilized and placed in contact with a solid growth medium suitable for inducing the formation of de-differentiated calli. Plant cell growth media are known in the art and comprise the nutrients and growth hormones required for the cells to multiply, as described in Hall (“Plant cell culture initiation.” Molecular biotechnology 16.2 (2000): 161-173). Once de-differentiated calli are formed, they may optionally be screened for the presence of desired compounds, e.g., gum base ingredient components such as terpenoid compounds. Calli are then transferred to larger culture vessels, such as shake flasks, for the establishment of plant cell cultures a growth medium. Typically, plant cell cultures are established as suspension cultures in a liquid growth medium, which may be a liquid version of the same growth medium used for de-differentiation or a different growth medium.

An alternative method of plant cell line development comprises isolating cambial meristematic cell (CMCs) from the selected plant and establishing the CMCs in plant cell suspension cultures. Exemplary methods for the establish of CMCs are known in the art and described in, e.g., Lee et al. “Cultured cambial meristematic cells as a source of plant natural products.” Nature biotechnology 28.11 (2010): 1213-1217) and Roberts and Kolewe (“Plant natural products from cultured multipotent cells.” Nature biotechnology 28.11 (2010): 1175-1176).

Established plant cell cultures may be analyzed for the production of one or more components of the desired gum base ingredient (e.g., polyisoprene, triterpenoid acetates, terpenes, resin acids, or any combination thereof) using a suitable method. After the cells are allowed to grow in the culture medium, the medium may then be processed for downstream analysis of the gum base ingredient components, e.g., by cell lysis, extraction, or separation, methods of which are known to one of skill in the art. Suitable methods for analyzing the culture medium for gum base ingredient components may include, for example, thin layer chromatography (TLC), high-performance liquid chromatography (HPLC), ultra performance liquid chromatography (UPLC), gas chromatography-mass spectrometry (GC-MS), liquid chromatography-mass spectrometry (LC-MS), liquid chromatography tandem mass spec methods (HPLC/MS/MS or UPLC/MS/MS), gel permeation chromatography (GPC), Fourier transform infrared spectroscopy (FTIR), nuclear magnetic resonance (NMR) spectroscopy, differential scanning calorimetry (DSC), size exclusion chromatography, light scattering, or liquid chromatography at critical condition (LCCC). Plant cell lines that are found to produce one or more desired gum base components, either detectably or above a certain threshold titer in the culture medium (e.g., at least 0.01 mg/L, at least 0.1 mg/L, at least 0.5 mg/L, at least 1 mg/L, at least 5 mg/L, at least 10 mg/L, at least 50 mg/L, at least 100 mg/L, at least 0.5 g/L, at least 1 g/L, at least 5 g/L, or at least 10 g/L) may be selected for further development and scale up.

In order to minimize variability in downstream production processes, a cell bank of the desired cell line is generated and stored in cryopreservation (e.g., at −20° C., −80° C., or −120° C.) to serve as an inoculum for plant cell fermentation processes.

### Plant Cell Culture Development and Scale-Up

Once one or more suitable cell lines have been selected for plant cell cultures to produce the cultured gum base ingredient, the cell lines are cultured in a medium in a plant cell culture (also known as plant cell fermentation) process to produce the cultured gum base ingredient. The plant cell culture process may be developed and optimized to maximize production of the cultured gum base ingredient, to reduce costs associated with production of the gum base ingredient (e.g., cost of goods), or to increase the scale of cell culture process.

The production of the gum base ingredient may be scaled to any desired volume to suit various purposes. For example, for high to medium throughput screening of various culture conditions, the process may be scaled to take place in a microbioreactor (e.g., about 15 mL to about 500 mL) or a benchtop scale bioreactor e.g., ranging from about 0.5 L to about 15 L). Alternatively, the process may be scaled up to a pilot scale bioreactor (e.g., ranging from about 15 L to about 15,000 L), or a manufacturing scale bioreactor (e.g., about 15,000 L to about 75,000 L or greater). Any type of bioreactor known in the art may be used for plant cell culture for the production of cultured gum base ingredient, including, for example, a stirred-tank bioreactor, a pneumatic bioreactor (e.g., a bubble column or airlift bioreactor), a membrane bioreactor, a wave bioreactor, or a disposable bioreactor. Depending upon several factors, including the extent to which production of the gum base ingredient is (or not) associated with cell growth, a cell culture system may be selected to maximize the production of the gum base ingredient. A variety of cell culture systems are known in the art and include, for example, batch systems, semi-batch systems, fed batch systems, two-stage culture systems, perfusion culture systems, continuous systems, and semi-continuous systems. At each point during plant cell culture process development, cell cultures may be analyzed for the production of one or more components of the desired gum base ingredient (e.g., polyisoprene, triterpenoid acetates, terpenes, resin acids, or any combination thereof) using any suitable method known in the art or described herein.

Typically, optimization of plant cell culture parameters such as agitation (including, e.g., agitation method and speed), gas exchange (e.g., aeration rate and dissolved oxygen), temperature, light, pH, and run time are varied and tested at the microbioreactor or benchtop bioreactor scale to determine the optimal cell culture conditions for producing the cultured gum base ingredient. The plant cell cultures may be monitored using suitable analytical methods, such as those use for the initial characterization of the cell lines, as well as microscopy and flow cytometry to analyze cell morphology, cell aggregates (e.g., aggregate size distribution), single-cell analysis to measuring cell-to-cell variation, and molecular methods such as transcriptomics and proteomics (e.g., to measure variations in cell ploidy and gene expression). Methods known in the art may also be implemented for increasing the production titer of the gum base ingredient and/or for refining the composition of the gum base ingredient produced. One such method is elicitation, or the manipulation in the cell culture conditions to elicit a defense response in the plant cells, for example by adding jasmonates (e.g., jasmonic acid) to the cell culture. In some cases, especially those where accumulation of compounds in the cell culture result in toxicity to the plant cell or feedback inhibition, in situ product removal may be employed to increase the yield of desired product. In situ removal of the product may comprise the use of two-phase solid-liquid or immiscible liquid-liquid systems, or product adsorption onto a resin (e.g., a nonaromatic and/or microporous resin). In some cases, immobilization of plant cells in plant cell culture can lead to an increase in production of the desired product. Immobilization of plant cells may include, for example, encapsulation in, e.g., in situ removal of the product by two-phase solid-liquid or immiscible liquid-liquid systems or product adsorption onto a resin.

In some embodiments, the plant cell culture process is optimized such that the plurality of cells produces the cultured gum base ingredient at a certain threshold concentration. For example, in some embodiments, the plurality of plant cells produces at least 0.01 mg/L of cultured gum base ingredient in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of cultured gum base ingredient in the culture medium). In some embodiments which may be combined with the preceding embodiments, the plurality of plant cells produces at least 0.01 mg/L of terpenoid compounds in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of terpenoid compounds in the culture medium).

In some embodiments which may be combined with the preceding embodiments, the plurality of plant cells produces at least 0.01 mg/L of triterpenoid acetates in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of triterpenoid acetates in the culture medium). In some embodiments which may be combined with the preceding embodiments, the plurality of plant cells produces at least 0.01 mg/L of luperol acetate in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of luperol acetate in the culture medium). In some embodiments which may be combined with the preceding embodiments, the plurality of plant cells produces at least 0.01 mg/L of beta-amyrin acetate in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of beta-amyrin acetate in the culture medium). In some embodiments which may be combined with the preceding embodiments, the plurality of plant cells produces at least 0.01 mg/L of alpha-amyrin acetate in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of alpha-amyrin acetate in the culture medium). In some embodiments which may be combined with the preceding embodiments, the plurality of plant cells produces at least 0.01 mg/L of taraxasterol acetate in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of taraxasterol acetate in the culture medium).

In some embodiments which may be combined with the preceding embodiments, the plurality of plant cells produces at least 0.01 mg/L of polyterpenoids in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of polyterpenoids in the culture medium). In some embodiments which may be combined with the preceding embodiments, the plurality of plant cells produces at least 0.01 mg/L of polyisoprene in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of polyisoprene in the culture medium).

In some embodiments which may be combined with the preceding embodiments, the plurality of plant cells produces at least 0.01 mg/L of resin acids in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of resin acids in the culture medium). In some embodiments which may be combined with the preceding embodiments, the plurality of plant cells produces at least 0.01 mg/L of one or more abietic type acids in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of abietic type acids in the culture medium). In some embodiments which may be combined with the preceding embodiments, the plurality of plant cells produces at least 0.01 mg/L of one or more pimaric type acids in the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of pimaric type acids in the culture medium).

In some embodiments, the plurality of plant cells produces one, two, three, four, five, or all six of luperol acetate, beta-amyrin acetate, taraxasterol acetate, alpha-amyrin acetate, and polyisoprene, each independently at a titer of at least 0.01 mg/L of the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of the culture medium).

In some embodiments which may be combined with the preceding embodiments, the plurality of plant cells produces a polymer (e.g., a polyterpenoid, e.g., polyisoprene) having an average molecular weight of between about 100 Da and about 3,000,000 Da (e.g., between about 100 Da and about 100,000 Da or between about 10,000 Da and about 3,000,000 Da) at a concentration of at least 0.01 mg/L of the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of the culture medium). In certain embodiments which may be combined with the preceding embodiments, the plurality of plant cells produces a polymer (e.g., a polyterpenoid, e.g., polyisoprene) having an average molecular weight of between about 100 Da and about 3,000,000 Da (e.g., between about 100 Da and about 100,000 Da or between about 10,000 Da and about 3,000,000 Da) at a concentration of at least 0.01 mg/L of the culture medium (e.g., at least 0.01 mg/L, at least 0.1 mg/L, at least 0.5 mg/L, at least 1 mg/L, at least 5 mg/L, at least 10 mg/L, at least 25 mg/L, at least 50 mg/L, at least 75 mg/L, at least 100 mg/L, at least 200 mg/L, at least 300 mg/L, at least 400 mg/L, at least 0.5 g/L, at least 0.75 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 12.5 g/L, at least 15 g/L, at least 17.5 g/L, at least 20 g/L, at least 25 g/L, at least 30 g/L, at least 35 g/L, at least 40 g/L, at least 45 g/L, or at least 50 g/L of the culture medium).

### Concentrating, Isolating, and Purifying Cultured Gum Base Ingredient

Once the plant cell culture process produces a satisfactory titer of the gum base ingredient, the gum base ingredient may be recovered from the cell culture using a suitable method. In some embodiments, the methods described herein comprise concentrating the culture medium of the plant cell culture to produce a cultured gum base ingredient concentrate, and/or separating the cultured gum base ingredient from the culture medium or gum base ingredient concentrate to produce an isolated or purified cultured gum base ingredient.

As hydrophobic compounds, such as many terpenoids, are often stored in the cell wall, cell membranes, and organelles of plant cells (e.g., vesicles), recovery of the cultured gum base ingredient may include cell permeabilization and/or cell lysis. Numerous methods of cell permeabilization and lysis are known in the art and may include, for example, the use of cell wall digesting enzymes, homogenization, sonication, steam explosion, high pressure methods (e.g., French press), pH shock, chemical shock, and electroporation.

In some instances, the culture medium in which the plant cells have been cultured is concentrated (e.g., by removing water) to increase the concentration of the cultured gum base ingredient in the medium, to produce a cultured gum base ingredient concentrate. The culture medium may be concentrated using any suitable means known in the art, including, for example, boiling, distillation, osmosis (e.g., forward osmosis or reverse osmosis), and the like.

In some embodiments, the cultured gum base ingredient is separated from the culture medium from the plant cell culture, or the cultured gum base ingredient concentrate, to produce an isolated or purified cultured gum base ingredient. The separation may one step or two or more steps, each of which may be performed using any suitable technique known in the art. For example, initial separation of the cultured gum base ingredient may comprise extraction (e.g., liquid-liquid extraction), precipitation, adsorption, membrane separation, aqueous two-phase separation (ATPS), distillation, or a combination thereof. Following initial separation, the cultured gum base ingredient may be further isolated or purified via chromatography. Chromatographic methods for purifying products from cell culture are known in the art and include, for example, HPLC, UPLC, size-exclusion chromatography, and countercurrent chromatography. The cultured gum base ingredient may be purified to any suitable level (e.g., 80%, 81%, 82%, 83%, 84%, 85%, 86%, 87%, 88%, 89%, 90%, 91%, 92%, 93%, 94%, 95%, 96%, 97%, 98%, or 99% purity). The resulting isolated or purified cultured gum base ingredient may be further processed into a suitable form for use in preparing a gum base, e.g., a powder, a liquid, a solid mass, tablets, pellets, coated pellets, sticks, tabs, hollow balls, cubes, or chunks.

### Food Products Comprising Cultured Gum Base Ingredients

In some aspects, provided herein is a food product, and methods of making thereof, that comprises any of the cultured gum base ingredients described herein. In some embodiments, provided herein is a process for producing a food product (e.g., a gum base or a chewing gum), the process comprising a step of including any of the cultured gum base ingredients, cultured gum base ingredient concentrates, or isolated or purified cultured gum base ingredients described herein in the food product.

### Gum Bases

In some embodiments, the food product is a gum base. Accordingly, in some embodiments, provided herein is a process of producing a gum base, the method comprising a step of including any of the cultured gum base ingredients, cultured gum base ingredient concentrates, or isolated or purified cultured gum base ingredients described herein in the food product. As used herein, the term “gum base” typically refers an insoluble, non-nutritive, masticatory substance formulated to provide a pleasant chewing experience. The gum base serves as a carrier for the other ingredients in the chewing gum, such as flavorings, colorants, sweeteners, and the like, and is formulated provide gradual release of flavors and sweeteners. Gum bases suitable for producing desirable chewing gum typically have a complex viscosity of between 10⁴ and 10⁸ Pa*s at room temperature and between 10² and 10⁵ Pa*s at a temperature of 60° C. and above, as measured by linear viscoelastic (LVE) rheology. In some embodiments, the gum base has a complex viscosity of between about 10⁴ and about 10⁸ Pa*s (e.g., between about 10⁴ and about 10⁵ Pa*s, between about 10⁴ and about 10⁶ Pa*s, between about 10⁴ and about 10⁷ Pa*s, between about 10⁴ and about 10⁸ Pa*s, between about 10⁵ and about 10⁶ Pa*s, between about 10⁵ and about 10⁷ Pa*s, between about 10⁵ and about 10⁸ Pa*s, between about 10⁶ and about 10⁷ Pa*s, between about 10⁶ and about 10⁸ Pa*s, or between about 10⁷ and about 10⁸ Pa*s) at a temperature of between about 15° C. and about 25° C. (e.g., about 15° C., about 16° C., about 17° C., about 18° C., about 19° C., about 20° C., about 21° C., about 22° C., about 23° C., about 24° C., or about 25° C.) and between 10² and 10⁵ Pa*s (e.g., between about 10² and about 10³ Pa*s, about 10² and about 10⁴ Pa*s, about 10² and about 10⁵ Pa*s, about 10³ and about 10⁴ Pa*s, about 10³ and about 10⁵ Pa*s, or about 10⁴ and about 10⁵ Pa*s) at a temperature of at least about 60° C. (e.g., at least about 60° C., at least about 65° C., at least about 70° C., at least about 75° C., at least about 80° C., at least about 85° C., at least about 90° C., or higher), as measured by linear viscoelastic (LVE) rheology. Methods for measuring complex viscosity, including by LVE rheology, are known in the art and described herein and, e.g., in Rheology: Principles, Measures, and Applications by Christopher W. Macosko, WILEY-VCH, 1^{st} edition, 1996.

In some embodiments, the gum base comprises any of the cultured gum base ingredients described herein in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises any of the cultured gum base ingredients described herein in an amount of between about 1% and about 25% (e.g., between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base.

In some embodiments, the gum base comprises terpenoid compounds in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises terpenoid compounds in an amount of between about 1% and about 25% (e.g., between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base.

In some embodiments, the gum base comprises terpenoid acetates in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises terpenoid acetates in an amount of between about 0.1% and about 25% (e.g., between about 0.1% and about 1%, between about 0.1% and about 5%, between about 0.1% and about 10%, between about 0.1% and about 15%, between about 0.5% and about 1%, between about 0.5% and about 5%, between about 0.5% and about 10%, between about 0.5% and about 15%, between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base. In some embodiments, the gum base comprises one, two, three, or all four of luperol acetate, beta-amyrin acetate, taraxasterol acetate, and alpha-amyrin acetate, each independently in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises In some embodiments, the gum base comprises one, two, three, or all four of luperol acetate, beta-amyrin acetate, taraxasterol acetate, and alpha-amyrin acetate, each independently in an amount of between about 0.1% and about 25% (e.g., between about 0.1% and about 1%, between about 0.1% and about 5%, between about 0.1% and about 10%, between about 0.1% and about 15%, between about 0.5% and about 1%, between about 0.5% and about 5%, between about 0.5% and about 10%, between about 0.5% and about 15%, between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base.

In some embodiments, the gum base comprises polyterpenoids in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises polyterpenoids in an amount of between about 0.1% and about 25% (e.g., between about 0.1% and about 1%, between about 0.1% and about 5%, between about 0.1% and about 10%, between about 0.1% and about 15%, between about 0.5% and about 1%, between about 0.5% and about 5%, between about 0.5% and about 10%, between about 0.5% and about 15%, between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base. In some embodiments, the gum base comprises polyisoprene in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises In some embodiments, the gum base comprises polyisoprene in an amount of between about 0.1% and about 25% (e.g., between about 0.1% and about 1%, between about 0.1% and about 5%, between about 0.1% and about 10%, between about 0.1% and about 15%, between about 0.5% and about 1%, between about 0.5% and about 5%, between about 0.5% and about 10%, between about 0.5% and about 15%, between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base. In certain embodiments, the polyterpenoid (e.g., polyisoprene) has an average molecular weight of between about 100 Da and about 3,000,000 Da (e.g., between about 100 Da and about 100,000 Da or between about 10,000 Da and about 3,000,000 Da).

In some embodiments, the gum base comprises resin acids in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises resin acids in an amount of between about 0.1% and about 25% (e.g., between about 0.1% and about 1%, between about 0.1% and about 5%, between about 0.1% and about 10%, between about 0.1% and about 15%, between about 0.5% and about 1%, between about 0.5% and about 5%, between about 0.5% and about 10%, between about 0.5% and about 15%, between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base.

In some embodiments, the gum base comprises abietic type acids in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises abietic type acids in an amount of between about 0.1% and about 25% (e.g., between about 0.1% and about 1%, between about 0.1% and about 5%, between about 0.1% and about 10%, between about 0.1% and about 15%, between about 0.5% and about 1%, between about 0.5% and about 5%, between about 0.5% and about 10%, between about 0.5% and about 15%, between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base.

In some embodiments, the gum base comprises pimaric type acids in an amount of at least 0.1% (e.g., at least 0.1%, at least 0.5%, at least 1%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20%) by weight of the gum base. In some embodiments, the gum base comprises pimaric type acids in an amount of between about 0.1% and about 25% (e.g., between about 0.1% and about 1%, between about 0.1% and about 5%, between about 0.1% and about 10%, between about 0.1% and about 15%, between about 0.5% and about 1%, between about 0.5% and about 5%, between about 0.5% and about 10%, between about 0.5% and about 15%, between about 1% and about 5%, between about 1% and about 10%, between about 1% and about 15%, between about 1% and about 20%, between about 1% and about 25%, between about 5% and about 7.5%, between about 5% and about 10%, between about 5% and about 12.5%, between about 5% and about 15%, between about 5% and about 17.5%, between about 5% and about 20%, between about 5% and about 25%, between about 7.5% and about 10%, between about 7.5% and about 12.5%, between about 7.5% and about 15%, between about 7.5% and about 17.5%, between about 7.5% and about 20%, between about 7.5% and about 25%, between about 10% and about 12.5%, between about 10% and about 15%, between about 10% and about 17.5%, between about 10% and about 20%, between about 10% and about 25%, between about 12.5% and about 15%, between about 12.5% and about 17.5%, between about 12.5% and about 20%, between about 12.5% and about 25%, between about 15% and about 17.5%, between about 15% and about 20%, between about 15% and about 25%, between about 17.5% and about 25%, or between about 20% and about 25%) by weight of the gum base.

In some embodiments, the gum base comprises one, two, three, four, five, or all six of luperol acetate, beta-amyrin acetate, taraxasterol acetate, alpha-amyrin acetate, and polyisoprene. In some embodiments, the gum base comprises one, two, three, four, five, or all six of luperol acetate in an amount of between 1% and 15% by dry weight; beta-amyrin acetate in an amount of between 0.05% and 10% by dry weight; taraxasterol acetate in an amount of between 0.01% and 5% by dry weight; alpha-amyrin acetate in an amount of between 0.01% and 5% by dry weight; and polyisoprene in an amount of between 0.05% and 10% by dry weight. In certain embodiments, the gum base comprises one, two, three, four, five, or all six of luperol acetate in an amount of between 2% and 10% by dry weight; beta-amyrin acetate in an amount of between 0.5% and 5% by dry weight; taraxasterol acetate in an amount of between 0.05% and 3% by dry weight; alpha-amyrin acetate in an amount of between 0.1% and 3% by dry weight; and polyisoprene in an amount of between 0.5% and 5% by dry weight. In certain embodiments, the gum base comprises luperol acetate, beta-amyrin acetate, taraxasterol acetate, alpha-amyrin acetate, and polyisoprene in a ratio of about 9:3:1:1:3 by dry weight.

In some embodiments, the gum base comprises one or more additional components in addition to the cultured gum base ingredient. In some embodiments, the gum base comprises one, two, three, four, five, six, or all seven of filler component (e.g., talc, calcium carbonate, or a combination thereof) terpene resin, gum resin, polyvinyl acetate, vegetable fats, emulsifiers, waxes, and antioxidants. In some embodiments, the gum base does not comprise comprises one or more of talc, calcium carbonate, terpene resin, gum resin, polyvinyl acetate, vegetable fats, emulsifiers, waxes, and antioxidants. In certain embodiments, which may be combined with the preceding embodiments, the gum base comprises filler component (e.g., talc, calcium carbonate, or a combination thereof) in an amount of between about 0.01% and about 30% (e.g., between 0.01% and 30%, between 10% and 30%, between 15% and 25%, between 18% and 22%, or about 20%) by weight of the gum base. In certain embodiments, which may be combined with the preceding embodiments, the gum base comprises terpene resin, gum resin, or a combination thereof in an amount of between about 5% and about 50% (e.g., between 5% and 15%, between 8% and 12%, between 5% and 20%, between 5% and 25%, between 5% and 30%, between 5% and 40%, between 5% and 50%, between 10% and 25%, between 10% and 30%, between 10% and 35%, between 10% and 40%, between 10% and 50%, between 15% and 25%, between 18% and 22%, between 15% and 30%, between 15% and 35%, between 15% and 40%, between 15% and 50%, between 20% and 30%, between 23% and 27%, between 20% and 35%, between 20% and 40%, between 20% and 50%, between 25% and 35%, between 28% and 32%, between 25% and 40%, between 25% and 50%, between 30% and 40%, between 33% and 37%, between 30% and 50%, about 10%, about 15%, about 20%, about 25%, about 30%, about 35%, about 40%, or about 50%) by weight of the gum base. In certain embodiments, which may be combined with the preceding embodiments, the gum base comprises polyvinyl acetate in an amount of between about 0.01% and about 60% (e.g., between 0.01% and 60%, between 5% and 10%, between 5% and 15%, between 8% and 12%, between 5% and 20%, between 5% and 25%, between 5% and 30%, between 5% and 35%, between 5% and 45%, between 5% and 50%, between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 23% and 27%, between 20% and 35%, between 20% and 40%, between 20% and 45%, between 20% and 50%, between 25% and 30%, between 25% and 35%, between 28% and 32%, between 25% and 40%, between 25% and 45%, between 25% and 50%, between 30% and 35%, between 30% and 40%, between 30% and 45%, between 30% and 50%, between 35% and 40%, between 35% and 45%, between 38% and 42%, between 35% and 50%, between 40% and 45%, between 40% and 50%, between 43% and 47%, between 45% and 50%, about 10%, about 15%, about 20%, about 25%, about 30%, about 35%, about 40%, about 45%, or about 50%) by weight of the gum base. In certain embodiments, which may be combined with the preceding embodiments, the gum base comprises one or more vegetable fats in an amount of between about 5% and about 30% (e.g., between 5% and 30%, between 10% and 25%, between 10% and 20%, between 10% and 15%, between 12% and 16%, between 14% and 18%, between 16% and 20%, about 15%, about 16%, about 17%, about 18%, about 19%, or about 20%) by weight of the gum base. In certain embodiments, which may be combined with the preceding embodiments, the gum base comprises one or more emulsifiers in an amount of between about 0.01% and about 10% (e.g., between 0.01% and 10%, between 0.1% and 5%, between 0.5% and 5%, between 1% and 2%, between 1% and 3%, between 1% and 4%, between 1% and 5%, between 2% and 3%, between 2% and 4%, between 2% and 5%, between 3% and 4%, between 3% and 5%, between 4% and 5%, about 1%, about 0.95%, about 5%, or about 4.95%) by weight of the gum base. In certain embodiments, which may be combined with the preceding embodiments, the gum base comprises one or more waxes in an amount of between about 0.01% and about 15% (e.g., between 0.01% and 15%, between 1% and 10%, between 1% and 5%, between 3% and 7%, between 5% and 9%, between 6% and 10%, between 7% and 11%, about 5%, or about 9%) by weight of the gum base. In certain embodiments, which may be combined with the preceding embodiments, the gum base comprises one or more antioxidants in an amount of between about 0.01% and about 1% (e.g., between 0.01% and 1%, between 0.01% and 0.5%, between 0.01% and 0.1%, between 0.03% and 0.07%, or about 0.05%) by weight of the gum base.

In some embodiments, the gum base comprises the composition of any one of the gum base formulations as described in Tables 3A-3I. In certain embodiments, the gum base comprises the composition of the gum base designated as Exp 4, Exp 5, Exp 6, Exp 7, Exp 8, Exp 9, Exp 10, Exp 11, Exp 12, Exp 13, Exp 14, Exp 15, Exp 16, Exp 17, Exp 18, Exp 19, Exp 20, Exp 21, Exp 22, Exp 23, Exp 24, Exp 25, Exp 26, Exp 27, Exp 28, Exp 29, Exp 30, Exp 31, Exp 32, Exp 33, Exp 34, Exp 35, Exp 36, Exp 37, Exp 38, Exp 39, Exp 40, Exp 41, Exp 42, Exp 43, Exp 44, Exp 45, Exp 46, Exp 47, Exp 48, Exp 49, Exp 50, Exp 51, Exp 52, Exp 53, Exp 54, Exp 55, Exp 56, or Exp 57 in Tables 3A-3I.

### Chewing Gums

In some embodiments, the food product comprising the cultured gum base ingredient is a chewing gum. Therefore, in some aspects, provided herein is a process for producing a chewing gum, the process comprising a step of including any of the cultured gum base ingredients, cultured gum base ingredient concentrates, or isolated or purified cultured gum base ingredients described herein in the food product. In some embodiments, provided herein is a process for producing a chewing gum, the process comprising a step of including any of the gum bases comprising the cultured gum base ingredient described herein in the chewing gum. Accordingly, in some aspects, provided herein is a chewing gum comprising any of the cultured gum base ingredients, cultured gum base ingredient concentrates, or isolated or purified cultured gum base ingredients described herein.

In some embodiments, provided herein is a chewing gum comprising any of the gum bases described herein (e.g., any gum base described herein comprising the cultured gum base ingredient). In certain embodiments, the chewing gum comprises the gum base in an amount of between about 5% and about 95% (e.g., between 5% and 95%, between 5% and 10%, between 5% and 15%, between 5% and 20%, between 5% and 25%, between 5% and 30%, between 5% and 35%, between 5% and 40%, between 5% and 45%, between 5% and 50%, between 5% and 60%, between 5% and 70%, between 5% and 80%, between 5% and 90%, between 10% and 15%, between 10% and 20%, between 10% and 25%, between 10% and 30%, between 10% and 35%, between 10% and 40%, between 10% and 45%, between 10% and 50%, between 10% and 60%, between 10% and 70%, between 10% and 80%, between 10% and 90%, between 15% and 20%, between 15% and 25%, between 15% and 30%, between 15% and 35%, between 15% and 40%, between 15% and 45%, between 15% and 50%, between 15% and 60%, between 15% and 70%, between 15% and 80%, between 15% and 90%, between 20% and 25%, between 20% and 30%, between 20% and 35%, between 20% and 40%, between 20% and 45%, between 20% and 50%, between 20% and 60%, between 20% and 70%, between 20% and 80%, between 20% and 90%, between 25% and 30%, between 25% and 35%, between 25% and 40%, between 25% and 45%, between 25% and 50%, between 25% and 60%, between 25% and 70%, between 25% and 80%, between 25% and 90%, between 30% and 35%, between 30% and 40%, between 30% and 45%, between 30% and 50%, between 30% and 60%, between 30% and 70%, between 30% and 80%, between 30% and 90%, between 35% and 40%, between 35% and 45%, between 35% and 50%, between 35% and 60%, between 35% and 70%, between 35% and 80%, between 35% and 90%, between 40% and 45%, between 40% and 50%, between 40% and 60%, between 40% and 70%, between 40% and 80%, between 40% and 90%, between 45% and 50%, between 45% and 60%, between 45% and 70%, between 45% and 80%, between 45% and 90%, between 50% and 60%, between 50% and 70%, between 50% and 80%, between 50% and 90%, between 60% and 70%, between 50% and 80%, between 60% and 90%, between 70% and 80%, between 70% and 90%, or between 80% and 90%) by weight of the chewing gum.

In some embodiments which may be combined with previous embodiments, the chewing gum comprises of any one of the gum base formulations as described in Tables 3A-3I. In certain embodiments which may be combined with previous embodiments, the chewing gum comprises the gum base designated as Exp 4, Exp 5, Exp 6, Exp 7, Exp 8, Exp 9, Exp 10, Exp 11, Exp 12, Exp 13, Exp 14, Exp 15, Exp 16, Exp 17, Exp 18, Exp 19, Exp 20, Exp 21, Exp 22, Exp 23, Exp 24, Exp 25, Exp 26, Exp 27, Exp 28, Exp 29, Exp 30, Exp 31, Exp 32, Exp 33, Exp 34, Exp 35, Exp 36, Exp 37, Exp 38, Exp 39, Exp 40, Exp 41, Exp 42, Exp 43, Exp 44, Exp 45, Exp 46, Exp 47, Exp 48, Exp 49, Exp 50, Exp 51, Exp 52, Exp 53, Exp 54, Exp 55, Exp 56, or Exp 57 in Tables 3A-3I.

In some embodiments which may be combined with previous embodiments, all of the components of the chewing gum are derived from natural sources. In some variations, natural sources may include, for example, those sources that are derived from natural organisms, including, e.g., plants or plant cells. In certain embodiments which may be combined with previous embodiments, all of the components of the chewing gum are derived from plant sources. As used herein, plant sources may include, for example, plant cell culture and plant cell fermentation processes. In certain embodiments which may be combined with previous embodiments, the chewing gum is free of artificial and synthetic ingredients. In some variations, artificial and synthetic ingredients are ingredients that are made by chemical synthesis (e.g., petrochemicals) and are not derived from plant sources. Synthetic ingredients may include, for example, synthetic elastomers including polyisobutylene, butyl rubber, styrene copolymers, polyisoprene, polyethylene, and vinyl acetate-vinyl laurate copolymer. In certain embodiments, which may be combined with previous embodiments, the chewing gum is free of ingredients that are derived from petroleum.

Chewing gum compositions of the present disclosure may be made using a variety of different compositions that are typically used in chewing gum compositions. Suitable physical forms include sticks, tabs, drageés, chicklets, batons, and the like. Although exact ingredients for each product form will vary from product to product, the specific techniques will be known by one skilled in the art. In general, a chewing gum composition typically contains a chewable gum base portion which is essentially water-insoluble, and a water-soluble bulk portion which includes water soluble bulking agents and other water-soluble components as well as flavors and perhaps other active ingredients which are typically water-insoluble. The water-soluble portion dissipates with a portion of the flavor (and other water insoluble actives, if present) over a period of time during chewing. The gum base portion is retained in the mouth throughout the chew.

The chewing gum of the present disclosure may be a coated and/or sugarless chewing gum containing the cultured gum base ingredient. In certain embodiments of the present disclosure, the chewing gum is a coated chewing gum comprising a chewing gum center and a coating. Unless otherwise indicated, the amounts of chewing gum components set forth herein are by weight of the chewing gum center. The coating may partially or completely cover the surface of the chewing gum center.

The chewing gum center may comprise between approximately 5% to about 95% by weight gum base. Typically, the insoluble gum base may comprise between approximately 10% and about 50% by weight of the chewing gum center, or from approximately 20% to about 40% by weight of the chewing gum center. The present disclosure contemplates employing any commercially acceptable gum base formulation comprising the cultured gum base ingredient.

The insoluble gum base generally comprises elastomers, elastomer solvents, plasticizers, waxes, emulsifiers, and inorganic fillers. Plastic polymers, such as polyvinyl acetate, which behave somewhat as plasticizers, are also included. Other plastic polymers that may be used include polyvinyl laurate, polyvinyl alcohol, and polyvinyl pyrrolidone. Gum base typically comprises 20 to 40% by weight of the overall chewing gum composition (e.g., by weight of the chewing gum center). However, in less common formulations it may comprise as low as 5% or as high as 95% by weight.

Elastomers provide the rubbery texture which is characteristic of chewing gum. The elastomers used will depend on, for example, whether the chewing gum in which the base is used is adhesive or conventional, synthetic or natural, bubble gum or regular gum. Elastomers typically make up 5 to 25% by weight of the gum base. Synthetic elastomers may include, for example, polyisobutylene (e.g. having a weight average molecular weight of about 10,000 to about 95,000), butyl rubber (isobutylene-isoprene copolymer), styrene copolymers (having for example a styrene-butadiene ratio of about 1:3 to about 3:1), polyisoprene, polyethylene, vinyl acetate-vinyl laurate copolymer (having for example a vinyl laurate content of about 5% to about 50% by weight of the copolymer), and combinations thereof. In some embodiments, the chewing gum of the present disclosure does not comprise synthetic elastomers (e.g., does not comprise polyisobutylene, butyl rubber, styrene copolymers, polyisoprene, polyethylene, vinyl acetate-vinyl laurate copolymer, or any combination thereof). Natural elastomers may include, for example, natural rubbers such as smoke or liquid latex and guayule, as well as natural gums such as chicle, jelutong, lechi caspi, perillo, sorva, massaranduba balata, massaranduba chocolate, nispero, rosindinha, gutta hang kang and mixtures thereof. Natural elastomers include the cultured gum base ingredients that are described herein. In some embodiments, the chewing gum of the present disclosure comprises any of the cultured gum base ingredients described herein, which serves as a natural elastomer.

Elastomer solvents, which are sometimes referred to as elastomer plasticizers, may include, for example, natural rosin esters such as glycerol esters, or partially hydrogenated rosin, glycerol esters of polymerized rosin, glycerol esters of partially dimerized rosin, glycerol esters of rosin, pentaerythritol esters of partially hydrogenated rosin, methyl and partially hydrogenated methyl esters of rosin, pentaerthyritol esters of rosin, synthetics such as terpene resins, polylimonene and other polyterpenes and/or any suitable combination of the forgoing. Elastomer solvents are typically employed at levels of 5 to 30% by weight of the gum base.

Gum base plasticizers are sometimes referred to as softeners (but are not to be confused with water soluble softeners used in the water-soluble portion of the gum). Typically, these include fats and oils as well as waxes. Fats and oils are typically vegetable oils which are usually partially or fully hydrogenated to increase their melting point. Vegetable oils suitable for such use include oils of cottonseed, soybean, palm (including palm kernel), coconut, shea, castor, peanut, corn, rapeseed, canola, sunflower, cocoa and others. Less commonly used are animal fats such as milk fat, tallow and lard. Structured fats, which are essentially synthetically compounded glycerol esters (triglycerides) of fatty acids of varying chain lengths, offer an ability to carefully adjust the softening profile by use of short and medium chain fatty acids which are less commonly found in nature. Commonly employed waxes include paraffin, microcrystalline and natural waxes such as beeswax and carnauba. Microcrystalline waxes, especially those with a high degree of crystallinity, may be considered bodying agents or textural modifiers. Plasticizers are typically employed at a level of 5 to 40% by weight of the gum base.

Plastic polymers, such as polyvinyl acetate, which behave somewhat as plasticizers, are also commonly used. Other plastic polymers that may be used include polyvinyl laurate, polyvinyl alcohol, and polyvinyl pyrrolidone. Most gum bases incorporate polyvinyl acetate at a level of 5 to 40% by weight of the gum base.

The gum base typically also includes a filler component. The filler component is typically an inorganic powder such as calcium carbonate, ground limestone, magnesium carbonate, talc, silicate types such as aluminum and magnesium silicate, dicalcium phosphate, tricalcium phosphate, cellulose polymers, such as wood, combinations thereof and the like. The filler may constitute from 5% to about 50% by weight of the gum base. Occasionally, a portion of the filler may be added to the chewing gum mixture separately from the gum base.

Emulsifiers, which may also have plasticizing properties, assist in homogenizing and compatibilizing the different base components. Commonly used emulsifiers include mono- and diglycerides such as glycerol monostearate, lecithin, glycerol triacetate, glycerol monostearate, acetylated monoglycerides, fatty acids and combinations thereof. Emulsifiers are commonly used at a level of 1 to 10% by weight of the gum base.

Gum bases commonly contain optional additives such as other antioxidants and colors which serve their normal functions. Less commonly, flavors and sweeteners may be added to the gum base. These additives, if used, are typically employed at levels of about 1% or less by weight of the gum base.

The water-soluble portion of the chewing gum center may comprise softeners, sweeteners, flavoring agents, and combinations thereof as well as other optional ingredients. For example, the majority of the water-soluble portion of the chewing gum center will typically comprise a water-soluble, powdered carbohydrate which serves as a bulking agent. In sugar gums, this most often is sucrose although other sugars such as fructose, erythrose, dextrose (glucose), levulose, tagatose, galactose, trehalose, corn syrup solids and the like, alone or in any combination may also be used.

Generally, sugarless chewing gums will employ sugar alcohols (also called alditols, polyols or polyhydric alcohols) as bulking agents due to their benefits of low carcinogenicity, reduced caloric content and reduced glycemic values. Such sugar alcohols include sorbitol, mannitol, xylitol, hydrogenated isomaltulose, maltitol, erythritol, hydrogenated starch hydrolysate solids, and the like, alone or in any combination. Longer chain saccharides such as polydextrose and fructo-oligosaccharides are sometimes employed for their reduced caloric properties and other health benefits. The bulking agents typically comprise approximately 5% to about 95% by weight of the chewing gum center.

Softeners are added to the chewing gum in order to optimize the chewability and mouth feel of the gum. Softeners, also known in the art as plasticizers or plasticizing agents, generally constitute between approximately 0.5% to about 15% by weight of the chewing gum center. These include glycerin, propylene glycol and aqueous sweetener solutions (syrups). Examples of syrups include corn syrups and (generically) glucose syrups which are usually prepared from hydrolyzed starch. For sugarless products, the starch hydrolysate may be hydrogenated to produce an ingredient known as hydrogenated starch hydrolysate syrups or maltitol syrups. These HSH syrups have largely replaced sorbitol solutions previously used in sugarless gums because they also function as binders to improve the flexibility and other physical properties of the gum. Softeners are also often used to control the humectancy (water absorbing properties) of the product.

An emulsifier is sometimes added to the gum to improve the consistency and stability of the gum product. They may also contribute to product softness. Lecithin is the most commonly employed emulsifier, although nonionic emulsifiers such as polyoxyethylene sorbitan fatty acid esters and partial esters of common fatty acids (lauric, palmitic, stearic and oleic acid hexitol anhydrides (hexitans and hexides) derived from sorbitol may also be used. When used, emulsifiers typically comprised 0.5 to 2% by weight of the chewing gum center.

The chewing gum may comprise one or more surface active agents, which can be salts of potassium, ammonium, or sodium. Sodium salts include anionic surface active agents, such as alkyl sulfates, including sodium lauryl sulfate, sodium laureth sulfate, and the like. Other sodium salts include sodium lauroyl sarcosinate, sodium brasslate, and the like. Suitable ammonium salts include betaine derivatives such as cocamidopropyl betaine, and the like.

In the case of sugarless gums, it is usually desirable to add high intensity sweeteners to compensate for the reduced sweetness resulting from substitution of sugar alcohols for the sucrose in sugar gums. More recently, the trend has been to also add high intensity sweeteners to sugar gums to boost and extend flavor and sweetness. High intensity sweeteners (which are sometimes called high potency or artificial sweeteners) may be defined as food acceptable chemicals which are at least twenty times sweeter than sucrose. Commonly used high intensity sweeteners include aspartame, sucralose, and acesulfame-K. Less common are saccharin, thaumatin, alitame, neotame, cyclamate, perilla derived sweeteners, stevia derived sweeteners, monatin, monellin and chalcones.

Usage levels for high intensity sweeteners may vary widely depending on the potency of the sweetener, local market preferences and the nature and level of other ingredients which might impart bitterness to the gum. Typical levels can range from about 0.01% to about 2% by weight, although some applications may dictate usage outside that range. These sweeteners may be combined together, or with non-high intensity sweeteners at varying levels to impart a sweetness synergy to the overall composition.

Flavors can also optionally be employed to impart a characteristic aroma and taste sensation to chewing gum products. As discussed herein, most flavors are water insoluble liquids but water soluble liquids and solids are also known. These flavors may be natural or artificial (synthetic) in origin. Often natural and artificial flavors are combined. It is also common to blend different flavors together in pleasing combinations. Although the range of flavors usable in chewing gums is nearly limitless, they commonly fall into several broad categories. Fruit flavors include lemon, orange, lime, grapefruit, tangerine, strawberry, apple, cherry, raspberry, blackberry, blueberry, banana, pineapple, cantaloupe, muskmelon, watermelon, grape, currant, mango, kiwi and many others as well as combinations. Mint flavors include spearmint, peppermint, wintergreen, basil, corn mint, menthol and others and mixtures thereof. Spice flavors include cinnamon, vanilla, clove, chocolate, nutmeg, coffee, licorice, eucalyptus, ginger, cardamom and many others. Also used are herbal and savory flavors such as popcorn, chili, corn chip and the like. Flavors are typically employed at levels of 0.1 to 4% by weight of the finished gum product (e.g., coated chewing gum). In recent years there has been a trend toward increasing flavor levels to provide higher flavor impact.

It is common to co-dry and encapsulate flavors with various carriers and/or diluents. For example, spray-dried flavors using gum Arabic, starch, cyclodextrin or other carriers are often used in chewing gum for protection, controlled release, control of product texture and easier handling as well as other reasons. When flavors are in such forms, it will often be necessary to increase the usage level to compensate for the presence of the carriers or diluents.

The chewing gum of the present disclosure may employ various sensates. Generally, sensates may be any compounds that cause a cooling, heating, warming, tingling or numbing, for example, to the mouth or skin. Cooling agents are trigeminal stimulants that impart a cool sensation to the mouth, throat and nasal passages. The most widely known cooling agent is menthol, although this is often considered a flavor due to its aroma properties and the fact that it is a natural component of peppermint oil. More often, the term cooling agent refers to other natural or synthetic chemicals used to impart a cooling sensation with minimal aroma. Commonly employed cooling agents include ethyl p-menthane carboxamide and other N-substituted p-menthane carboxamides, N, 2, 3-trimethyl-2-isopropyl-butanamide and other acyclic carboxamides, menthyl glutarate (Flavor Extract Manufacturing Association (FEMA 4006)), 3-1-menthoxypropane-1,2-diol, isopulegol, menthyl succinate, menthol propylene glycol carbonate, menthol ethylene glycol carbonate, menthyl lactate, menthyl glutarate, menthone glyceryl ketal, p-menthane-1, 8-diol, menthol glyceryl ether, N-tertbutyl-p-menthane-3-carboxamide, p-menthane-3-carboxylic acid glycerol ester, methyl-2-isopryl-bicyclo (2.2.1), heptane-2-carboxamide, menthol methyl ether and others and combinations thereof.

Cooling agents may be employed to enhance the cool taste of mint flavors or to add coolness to fruit and spice flavors. Cooling agents also provide the perception of breath freshening, which is the basis of the marketing of many chewing gums and confections.

Trigeminal stimulants other than cooling agents may be employed in the chewing gums of the present disclosure. These include warming agents such as capsaicin, capsicum oleoresin, red pepper oleoresin, black pepper oleoresin, piperine, ginger oleoresin, gingerol, shoagol, cinnamon oleoresin, cassia oleoresin, cinnamic aldehyde, eugenol, cyclic acetal of vanillin, menthol glycerin ether and unsaturated amides and tingling agents such as Jambu extract, vanillyl alkyl ethers such as vanillyl n-butyl ether, spilanthol, Echinacea extract and Northern Prickly Ash extract. Some of these components are also used as flavoring agents.

Chewing gum generally conveys oral care benefits. In addition to mechanical cleaning of the teeth provided by the chewing action, saliva stimulated by chewing, flavor and taste from the product conveys additional beneficial properties in reducing bad breath, neutralizing acid, and the like. Saliva also contains beneficial polypeptides and other components which may improve the oral environment. These include: antimicrobial proteins, such as lysozyme, lactoferrin, peroxidases, and histatins; inhibitors of spontaneous crystallization, such as statherin.

The chewing gums of the present disclosure can provide these benefits along with the benefits disclosed herein, and may also be used as vehicles for the delivery of specialized oral care agents. These may include antimicrobial compounds such as Cetylpyridinium Chloride (CPC), triclosan and chlorhexidine; anti-caries agents such as calcium and phosphate ions, plaque removal agents such as abrasives, surfactants and compound/ingredients; plaque neutralization agents such as ammonium salts, urea and other amines; anti-tartar/calculus agents such as soluble pyrophosphates salts; anti halitosis agents such as parsley oil and copper or zinc salts of gluconic acid, lactic acid, acetic acid or citric acid, and whitening agents such as peroxides; agents that may provide either local or systemic anti-inflammatory effects to limit gingivitis, such as COX-2 inhibitors; agents that may reduce dentinal hypersensitivity, such as potassium salts to inhibit nerve cell transmission, and calcium phosphate salts to block the dentinal tubules.

Certain flavors such as peppermint, methyl salicylate, thymol, eucalyptol, cinnamic aldehyde and clove oil (eugenol) may have antimicrobial properties which benefit the oral cavity. These flavors may be present primarily for flavoring purposes or may be added specifically for their antimicrobial properties.

Certain mineral agents may contribute to dental health, in addition to ones disclosed by the disclosure by combating demineralization and enhancing remineralization of teeth. Such ingredients include fluoride salts, dental abrasives and combinations thereof.

Teeth color modifying substances may be considered among the oral care actives useful. These substances are suitable for modifying the color of the teeth to satisfy the consumer such as those listed in the CTFA Cosmetic Ingredient Handbook, 3rd Edition, Cosmetic and Fragrances Associations Inc., Wash. D. C. (1982), incorporated herein by reference. Specific examples include talc, mica, magnesium carbonate, magnesium silicate, aluminum magnesium carbonate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder and mixtures thereof.

The chewing gums of the present disclosure may be used to deliver biologically active agents and other beauty ingredients to the chewer. Such ingredients include vitamins, minerals, antioxidants, nutritional supplements, dietary supplements, functional food ingredients (e.g., probiotics, prebiotics, lycopene, phytosterols, stanol/sterol esters, omega-3 fatty acids, adenosine, lutein, zeaxanthin, grape seed extract, Ginkgo biloba, isothiocyanates and the like), OTC and prescription pharmaceuticals, vaccines, and nutritional supplements. It may be desirable to take certain steps to increase or decrease the rate of the release of the agent or to ensure that at least a minimum quantity is released. Such measures as encapsulation, isolation of the active, measures to increase or decrease interaction with the water-insoluble portion of the gum and enteric coating of actives may be employed to that end.

In general, chewing gum is manufactured by sequentially adding the various chewing gum ingredients to a commercially available mixer known in the art. After the ingredients have been thoroughly mixed, the gum mass is discharged from the mixer and shaped into the desired form such as rolling sheets and cutting into sticks, extruding into chunks or casting into pellets, which are then coated or panned.

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent. Further parts of the bulking agent are added to the mixer. Additional flavoring agents may be added with the final portion of the bulking agent or with the high intensity sweeteners. Other optional ingredients are added to the batch in a typical fashion, well known to those of ordinary skill in the art. The entire mixing procedure typically takes from five to fifteen minutes, but longer mixing times may sometimes be required. Those skilled in the art will recognize that many variations of the above described procedure may be followed.

Chewing gum base and chewing gum product have been manufactured conventionally using separate mixers, different mixing technologies and, often, at different factories. One reason for this is that the optimum conditions for manufacturing gum base, and for manufacturing chewing gum from gum base and other ingredients such as sweeteners and flavors, are so different that it has been impractical to integrate both tasks. Chewing gum base manufacturing involves the dispersive (often high shear) mixing of difficult-to-blend ingredients, such as elastomer, filler, elastomer plasticizer, base softeners/emulsifiers, and sometimes waxes. This process typically requires long mixing times. Chewing gum product manufacture also involves combining the gum base with more delicate ingredients such as product softeners, bulk sweeteners, high intensity sweeteners and flavoring agents using distributive (generally lower shear) mixing, for shorter periods.

Chewing gums of the present disclosure may also be coated. Pellet or ball gum is prepared as conventional chewing gum, but formed into pellets that are pillow shaped, or into balls. The pellets/balls can be then sugar coated or panned by conventional panning techniques to make a unique sugar coated pellet gum. Conventional panning procedures generally coat with sucrose, but recent advances in panning have allowed the use of other carbohydrate materials to be used in the place of sucrose. Some of these components include, for example, dextrose, maltose, palatinose, xylitol, lactitol, hydrogenated isomaltulose and other new alditols or a combination thereof. These materials may be blended with panning modifiers including, for example, gum arabic, maltodextrins, corn syrup, gelatin, cellulose type materials like carboxymethyl cellulose or hydroxymethyl cellulose, starch and modified starches, vegetable gums like alginates, locust bean gum, guar gum, and gum tragacanth, insoluble carbonates like calcium carbonate or magnesium carbonate and talc. Antitack agents may also be added as panning modifiers which allow the use of a variety of carbohydrates and sugar alcohols to be used in the development of new panned or coated gum products. Essential oils may also be added with the sugar coating to yield unique product characteristics.

### EXAMPLES

The presently disclosed subject matter will be better understood by reference to the following Examples, which are provided as exemplary of the invention, and not by way of limitation.

### Example 1: Production of Cultured Gum Base Ingredient by Plant Cell Fermentation

This Example describes the development of plant cell lines for the production of cultured gum base ingredient by plant cell fermentation (PCF). One or more plant cell lines are developed from plants that naturally produce gum base ingredients, such as Manilkara chicle, which produces chicle. Methods of developing plant cell lines for plant cell fermentation processes, as well as process optimization and scale-up, are described in, e.g., Andrews and Roberts (“Bioprocess engineering of plant cell suspension cultures.” Applied Bioengineering: Innovations and Future Directions 5 (2017)); Constabel (“Principles underlying the use of plant cell fermentation for secondary metabolite production.” Biochemistry and Cell Biology 66.6 (1988): 658-664); Dunwell (Plant Cell Culture—Present and Future, Plant Cell Culture: Essential Methods, 2010); Häkkinen et al. (“Plant cell cultures as food—aspects of sustainability and safety.” Plant cell reports 39.12 (2020): 1655-1668); Hellwig et al. (“Plant cell cultures for the production of recombinant proteins.” Nature biotechnology 22.11 (2004): 1415-1422); Taticek, Young, and Legge (“The scale-up of plant cell culture: Engineering considerations.” Plant cell, tissue and organ culture 24.2 (1991): 139-158); Wilson and Roberts (“Recent advances towards development and commercialization of plant cell culture processes for the synthesis of biomolecules.” Plant biotechnology journal 10.3 (2012): 249-268); and Xu and Zhang (“On the way to commercializing plant cell culture platform for biopharmaceuticals: present status and prospect.” Pharmaceutical bioprocessing 2.6 (2014): 499).

### Cell Line Development

In an exemplary plant cell line development method, one or more source plants are selected based on availability, genetic diversity, and the ability of the whole plants to generate a desired cultured gum base ingredient. Plant cell lines are developed from one or more plant species naturally produce a chicle, rubber, resin (e.g., rosin), or other type of latex suitable as a gum base ingredient, for example, Abies amabilis, Abies balsamea, Apios americana, Brosmium utile, Castilla fallax, Cnidoscolus elasticus, Cnidoscolus tepiquensis, Couma macrocarpa, Dyera costulata, Dyera lowii, Ficus platyphylla, Hevea brasilensis, Isonandra gutta, Lacmellea standleyi, Liquidambar orientalis, Liquidambar styraciflua, Manilkara chicle, Manilkara solimoesensis, Palaquium gutta, Palaquium leiocarpum, Parthenium argentatum, Picea rubens, Picea stichensis, Pinus ponderosa, Pinus lambertiana, Pistacia lentiscus, Taraxacum kok-saghyz. Explants (i.e., plant tissue samples) from a whole plant of the desired species are obtained from one or more tissues of the whole plant, for example from a bud (e.g., an apical bud and/or a lateral bud), a leaf or leaf tissue (e.g., a leaf primordium, a petiole, a leaf blade, leaf dermal tissue (e.g., cuticle and/or epidermis), leaf ground tissue (e.g., palisade mesophyll and/or spongy mesophyll), and/or leaf vascular tissue (e.g., xylem and/or phloem)), a stem (e.g., a tree trunk) or stem tissue (e.g., a node, an internode, bark or bark tissues, stem dermal tissue (e.g., epidermis), stem ground tissue (e.g., cortex and/or pith), stem vascular tissue (e.g., xylem and/or phloem), and/or stem meristematic tissue (e.g., vascular cambium)), or a root or root tissue (e.g., e.g., a taproot, a lateral root, a root apex, a root cap, root dermal tissue (e.g., root hairs and/or epidermis), root ground tissue (e.g., cortex, endodermis, and/or pericycle), root vascular tissue (e.g., xylem and/or phloem), and/or root meristematic tissue (e.g., vascular cambium)).

Once obtained, the explants are sterilized and used to inoculate a liquid, solid, or semi-solid cell growth medium suitable for the formation of calli of de-differentiated plant cells. Once de-differentiated calli are formed, they are transferred to larger culture vessels for the establishment of plant cell suspension cultures. Alternatively, cambial meristematic cell (CMCs) are isolated from the selected plant and established in laboratory-scale plant cell suspension cultures. The laboratory-scale plant cell suspension cultures are then analyzed for the production of one or more components of the desired gum base ingredient (e.g., polyisoprene, triterpenoid acetates, terpenes, resin acids, or any combination thereof) using a suitable method, such as thin layer chromatography (TLC), high-performance liquid chromatography (HPLC), ultra performance liquid chromatography (UPLC), gas chromatography-mass spectrometry (GC-MS), liquid chromatography-mass spectrometry (LC-MS), liquid chromatography tandem mass spec methods (HPLC/MS/MS or UPLC/MS/MS), gel permeation chromatography (GPC), Fourier transform infrared spectroscopy (FTIR), nuclear magnetic resonance (NMR) spectroscopy, differential scanning calorimetry (DSC), size exclusion chromatography, light scattering, or liquid chromatography at critical condition (LCCC). Optionally, the laboratory-scale suspension cultures are extracted or otherwise processed prior to analysis for the gum base ingredient components. Any plant cell line that is found to produce one or more desired gum base components, either detectably or above a certain threshold titer, is selected for further development and scale up.

In order to minimize variability in downstream production processes, a cell bank of the desired cell line is generated and stored in cryopreservation (e.g., at −20° C., −80° C., or −120° C.) to serve as an inoculum for plant cell fermentation processes.

### Plant Cell Culture Process Development

Once one or more suitable cell lines have been selected, the cell lines are cultured in a medium in a plant cell fermentation process to produce the cultured gum base ingredient. The production of the gum base ingredient is scaled to an appropriate volume for production such as a microbioreactor (e.g., about 15 mL to about 500 mL), a benchtop scale bioreactor (e.g., ranging from about 0.5 L to about 15 L), a pilot scale bioreactor (e.g., ranging from about 15 L to about 15,000 L), or a manufacturing scale bioreactor (e.g., about 15,000 L to about 75,000 L or greater). Process parameters such as agitation, gas exchange, temperature, light, and run time are varied and tested to determine the optimal cell cultured conditions. During process development and scaleup, the plant cells are cultured in one or more suitable bioreactor systems, such as a stirred-tank bioreactor, a pneumatic bioreactor (e.g., a bubble column or airlift bioreactor), a membrane bioreactor, a wave bioreactor, or a disposable bioreactor. An optimal bioreactor production system is selected or designed to maximize the production of the gum base ingredient, such as a batch system, a semi-batch system, a fed batch system, a two-stage culture system, a perfusion culture system, a continuous system, a semi-continuous system, or any suitable combination thereof. The production of the gum base ingredient by the cell suspension culture is monitored using suitable analytical methods, such as those use for the initial characterization of the cell lines, as well as microscopy and flow cytometry to analyze cell aggregates, single-cell analysis to measuring cell-to-cell variation, and molecular methods such as transcriptomics and proteomics. If needed, methods are implemented for increasing the production titer of the gum base ingredient and/or for refining the composition of the gum base ingredient produced, such as elicitation or induction of production by the addition of one or more elicitor compounds (e.g., jasmonate); in situ removal of the product by two-phase solid-liquid or immiscible liquid-liquid systems or product adsorption onto a resin; or immobilization of the plant cells, e.g., by encapsulation in agar, agarose, alginate, carrageenan, gelatin, hollow fiber membranes, polyacrylamide, or polyurethan foam. Once the plant cell suspension culture produces a satisfactory titer of the gum base ingredient, the gum base ingredient is recovered from the cell suspension culture using a suitable method, which may include cell lysis, extraction (e.g., liquid-liquid extraction), distillation, aqueous two-phase separation (ATPS), precipitation, adsorption, membrane separation, chromatography (e.g., HPLC, UPLC, size-exclusion chromatography, or countercurrent chromatography), or any combination thereof. The resulting cultured gum base ingredient is concentrated, isolated, purified, and/or crystalized to any suitable purity level and may be further processed into a suitable format for use in preparing a gum base, e.g., a powder, a liquid, a solid mass, or pellets.

### Example 2: Characterization of Cultured Gum Base Ingredient

This Example describes analysis and characterization of the composition and rheological property of the gum base ingredient produced in the cell culture process of Example 1.

The polymer or the mix of polymers and resins extracted from the plant cell culture in Example 1 (i.e., the cultured gum base ingredient) is fully characterized to determine its composition, molecular weight, and thermal mechanical properties. The characterization includes compositional analysis to determine the identity, relative quantity, and/or molecular weight of its components using one or more suitable methods for analyzing the compounds present (e.g., terpenoid compounds), such as TLC, HPLC, UPLC, GC-MS, LC-MS, HPLC/MS/MS, UPLC/MS/MS, GPC, FTIR, NMR spectroscopy, DSC, size exclusion chromatography, light scattering, or LCCC. Where the cultured gum base ingredient comprises a large hydrocarbon polymer, such as a polyisoprene, the molecular weight of the polymer may be determined by GPC, size exclusion chromatography, light scattering, viscometry, osmometry, or any combination thereof.

In addition to compositional analysis, the cultured gum base ingredient is analyzed to measure its rheological properties, such as its complex viscosity. Complex viscosity is a beneficial characteristic to compare for polymers considered for use in gum bases. The complex viscosity is the complex modulus divided by the angular frequency and represents the overall resistance to deformation of a material, regardless of whether that deformation is recoverable (elastic) or non-recoverable (viscous). Complex viscosity may be measured by linear viscoelastic rheology by a suitable means using commercially available instruments, for example, using the methods described in U.S. Pat. No. 10,492,512, which is hereby incorporated herein in its entirety.

In an exemplary method for characterizing the rheology of the gum base ingredient, one or more of the molecular weight, polydispersity (determined by GPC), glass transition temperature (determined by DSC), thermal degradation (temperature at which 1% of the sample was lost during thermal gravimetric analysis), complex viscosity (Pa*s), glass transition temperature (Tg), and shear modulus (G*), of the cultured gum base ingredient is measured using a suitable means known in the art. The properties are measured at various temperatures, including, e.g., room temperature (between ˜20° C. and ˜25° C.), mouth temperature (˜37° C.), and well above mouth temperature (e.g., ˜60° C. or higher), including the processing temperature (e.g., ˜120° C.).

### Example 3: Production and Characterization of Gum Base Comprising Cultured Gum Base Ingredient

In this Example, a gum base is made which comprises the cultured gum base ingredient produced according to Example 1 is used to produce one or more gum bases. Exemplary ranges for ingredients to be incorporated into the gum base are shown in Table 1. The ingredients are mixed and heated using any suitable method to produce the gum base, such as by mixing and heating (e.g., to 120° C.) either by hand or in a commercially available mixer at any appropriate scale.

TABLE 1
Exemplary gum base ingredients

| Ingredient | Amount (w/w %) |
|---|---|
| Cultured gum base ingredient | 5-20% |
| Talc | 0-20% |
| Calcium carbonate | 0-20% |
| Terpene resin | 10-35% |
| Polyvinyl acetate | 0-45% |
| Vegetable fats | 15-20% |
| Emulsifier | 0.95-4.95% |
| Wax | 0-10% |
| Antioxidant | ~0.05% |

### Example 4: Characterization and Optimization of Gum Base Comprising Cultured Gum Base Ingredient

This Example describes the characterization and optimization of the rheological properties of the gum base produced in Example 3.

An ideal gum base formulation should undergo a glass transition when it is heated from room temperature to mouth temperature. Gum bases with the desired glass transition typically have a complex viscosity of between 10⁴ and 10⁸ Pa*s at a temperature of between 20° C. and 25° C. (i.e., room temperature), and between 10² and 10⁵ Pa*s at a temperature of 60° C. and above. In addition to measuring complex viscosity, the properties of the gum base are evaluated by, e.g., visual inspection and measuring the glass transition temperature (Tg) and shear modulus (G*). Methods for measuring the glass transition temperature (Tg) and shear modulus (G*) of a gum base are provided in Methods for measuring the complex viscosity, glass transition temperature, and shear modulus of a gum base are U.S. Pat. No. 10,492,512, which is hereby incorporated herein in its entirety.

Optimization of the gum base formulation begins with the cultured gum base ingredient produced in Example 1, which may be a single compound or a blend of compounds extracted from plant cell fermentation, which may include, e.g., polyisoprene, triterpenoid acetates, terpenes, rosins, or a blend of any. To achieve the rheological properties defined above, a set of experiments are designed to select resin(s) (e.g., terpene resins and/or gum resins) to be blended with the cultured gum base ingredient, to determine the ratio of the cultured gum base ingredient to the resin, to select softeners and texture modifiers, and to improve stability as needed. Additional resins, processing aids, softeners, emulsifiers and stabilizers will be selected in sequence to the starting material to tune its LVE complex viscosity with desired compatibility, which can also be measured by visual inspection, changes in glass transition temperature (Tg), and shear modulus (G*).

Mixtures of cultured gum base ingredient and resin are prepared as shown in Table 2. The mixtures of Table 2 (Exp 1, Exp 2, and Exp 3) are then used to generate a variety of gum base formulations as described in Tables 3A-3I. Except for the mixtures of cultured gum base ingredient (Exp 1, Exp 2, and Exp 3), the ingredients to be used in the gum base formulations are obtained from commercial sources. The gum bases are produced as described in Example 3. The gum base formulations are then analyzed for LVE complex viscosity with desired compatibility as measured by visual inspection, changes in glass transition temperature (Tg), and shear modulus (G*), and optionally other parameters, as described in Example 3.

TABLE 2
Mixtures of cultured gum base ingredient and resin.
Amounts of ingredients are shows as percentages.

| | Exp 1 | Exp 2 | Exp 3 |
|---|---|---|---|
| Cultured Gum Base Ingredient | 95 | 80 | 70 |
| Resin (e.g., terpene resin | 5 | 20 | 30 |
| and/or gum resin) | | | |

TABLE 3A
Gum base formulations of mixture Exp 1 with varying
amounts of terpene resin and polyvinyl acetate. Amounts
of ingredients are shown as percentages.

| | Exp 4 | Exp 5 | Exp 6 | Exp 7 | Exp 8 | Exp 9 |
|---|---|---|---|---|---|---|
| Exp 1 | 10 | 15 | 5 | 10 | 15 | 5 |
| Talc | 20 | 20 | 20 | | | |
| Calcium | | | | 20 | 20 | 20 |
| Carbonate | | | | | | |
| Terpene Resin | 20 | 30 | 10 | 20 | 30 | 10 |
| Polyvinyl Acetate | 25 | 10 | 40 | 25 | 10 | 40 |
| Vegetable Fats | 15 | 15 | 15 | 15 | 15 | 15 |
| Emulsifier | 4.95 | 4.95 | 4.95 | 4.95 | 4.95 | 4.95 |
| Wax | 5 | 5 | 5 | 5 | 5 | 5 |
| Antioxidant | 0.05 | 0.05 | 0.05 | 0.05 | 0.05 | 0.05 |
| Total | 100 | 100 | 100 | 100 | 100 | 100 |

TABLE 3B
Gum base formulations of mixture Exp 2 with varying
amounts of terpene resin and polyvinyl acetate. Amounts
of ingredients are shown as percentages.

| | Exp 10 | Exp 11 | Exp 12 | Exp 13 | Exp 14 | Exp 15 |
|---|---|---|---|---|---|---|
| Exp 2 | 10 | 15 | 5 | 10 | 15 | 5 |
| Talc | 20 | 20 | 20 | | | |
| Calcium | | | | 20 | 20 | 20 |
| Carbonate | | | | | | |
| Terpene Resin | 20 | 30 | 10 | 20 | 30 | 10 |
| Polyvinyl Acetate | 25 | 10 | 40 | 25 | 10 | 40 |
| Vegetable Fats | 15 | 15 | 15 | 15 | 15 | 15 |
| Emulsifier | 4.95 | 4.95 | 4.95 | 4.95 | 4.95 | 4.95 |
| Wax | 5 | 5 | 5 | 5 | 5 | 5 |
| Antioxidant | 0.05 | 0.05 | 0.05 | 0.05 | 0.05 | 0.05 |
| Total | 100 | 100 | 100 | 100 | 100 | 100 |

TABLE 3C
Gum base formulations of mixture Exp 3 with varying
amounts of terpene resin and polyvinyl acetate. Amounts
of ingredients are shown as percentages.

| | Exp 16 | Exp 17 | Exp 18 | Exp 19 | Exp 20 | Exp 21 |
|---|---|---|---|---|---|---|
| Exp 3 | 10 | 15 | 5 | 10 | 15 | 5 |
| Talc | 20 | 20 | 20 | | | |
| Calcium | | | | 20 | 20 | 20 |
| Carbonate | | | | | | |
| Terpene Resin | 20 | 30 | 10 | 20 | 30 | 10 |
| Polyvinyl Acetate | 25 | 10 | 40 | 25 | 10 | 40 |
| Vegetable Fats | 15 | 15 | 15 | 15 | 15 | 15 |
| Emulsifier | 4.95 | 4.95 | 4.95 | 4.95 | 4.95 | 4.95 |
| Wax | 5 | 5 | 5 | 5 | 5 | 5 |
| Antioxidant | 0.05 | 0.05 | 0.05 | 0.05 | 0.05 | 0.05 |
| Total | 100 | 100 | 100 | 100 | 100 | 100 |

TABLE 3D
Gum base formulations of mixture Exp 1 with varying amounts
of vegetable fats, emulsifier, and wax. Amounts of
ingredients are shown as percentages.

| | Exp 22 | Exp 23 | Exp 24 | Exp 25 | Exp 26 | Exp 27 |
|---|---|---|---|---|---|---|
| Exp 1 | 20 | 20 | 20 | 20 | 20 | 20 |
| Talc | 20 | 20 | 20 | | | |
| Calcium | | | | 20 | 20 | 20 |
| Carbonate | | | | | | |
| Terpene Resin | 35 | 35 | 35 | 35 | 35 | 35 |
| Polyvinyl Acetate | 0 | 0 | 0 | 0 | 0 | 0 |
| Vegetable Fats | 15 | 19 | 15 | 15 | 19 | 15 |
| Emulsifier | 4.95 | 0.95 | 0.95 | 4.95 | 0.95 | 0.95 |
| Wax | 5 | 5 | 9 | 5 | 5 | 9 |
| Antioxidant | 0.05 | 0.05 | 0.05 | 0.05 | 0.05 | 0.05 |
| Total | 100 | 100 | 100 | 100 | 100 | 100 |

TABLE 3E
Gum base formulations of mixture Exp 2 with varying
amounts of vegetable fats, emulsifier, and wax. Amounts
of ingredients are shown as percentages.

| | Exp 28 | Exp 29 | Exp 30 | Exp 31 | Exp 32 | Exp 33 |
|---|---|---|---|---|---|---|
| Exp 2 | 20 | 20 | 20 | 20 | 20 | 20 |
| Talc | 20 | 20 | 20 | | | |
| Calcium | | | | 20 | 20 | 20 |
| Carbonate | | | | | | |
| Terpene Resin | 35 | 35 | 35 | 35 | 35 | 35 |
| Polyvinyl Acetate | 0 | 0 | 0 | 0 | 0 | 0 |
| Vegetable Fats | 15 | 19 | 15 | 15 | 19 | 15 |
| Emulsifier | 4.95 | 0.95 | 0.95 | 4.95 | 0.95 | 0.95 |
| Wax | 5 | 5 | 9 | 5 | 5 | 9 |
| Antioxidant | 0.05 | 0.05 | 0.05 | 0.05 | 0.05 | 0.05 |
| Total | 100 | 100 | 100 | 100 | 100 | 100 |

TABLE 3F
Gum base formulations of mixture Exp 3 with varying
amounts of vegetable fats, emulsifier, and wax. Amounts of
ingredients are shown as percentages.

| | Exp 34 | Exp 35 | Exp 36 | Exp 37 | Exp 38 | Exp 39 |
|---|---|---|---|---|---|---|
| Exp 3 | 20 | 20 | 20 | 20 | 20 | 20 |
| Talc | 20 | 20 | 20 | | | |
| Calcium | | | | 20 | 20 | 20 |
| Carbonate | | | | | | |
| Terpene Resin | 35 | 35 | 35 | 35 | 35 | 35 |
| Polyvinyl Acetate | 0 | 0 | 0 | 0 | 0 | 0 |
| Vegetable Fats | 15 | 19 | 15 | 15 | 19 | 15 |
| Emulsifier | 4.95 | 0.95 | 0.95 | 4.95 | 0.95 | 0.95 |
| Wax | 5 | 5 | 9 | 5 | 5 | 9 |
| Antioxidant | 0.05 | 0.05 | 0.05 | 0.05 | 0.05 | 0.05 |
| Total | 100 | 100 | 100 | 100 | 100 | 100 |

TABLE 3G
Gum base formulations of mixture Exp 1 with varying amounts
of mixture Exp1, terpene resin, polyvinyl acetate, and vegetable
fats. Amounts of ingredients are shown as percentages.

| | Exp 40 | Exp 41 | Exp 42 | Exp 43 | Exp 44 | Exp 45 |
|---|---|---|---|---|---|---|
| Exp 1 | 10 | 15 | 10 | 15 | 10 | 15 |
| Terpene Resin | 20 | 30 | 20 | 30 | 25 | 35 |
| Polyvinyl Acetate | 45 | 30 | 45 | 30 | 45 | 30 |
| Vegetable Fats | 15 | 15 | 20 | 20 | 20 | 20 |
| Emulsifier | 4.95 | 4.95 | 4.95 | 4.95 | 4.95 | 4.95 |
| Wax | 5 | 5 | | | | |
| Antioxidant | 0.05 | 0.05 | 0.05 | 0.05 | 0.05 | 0.05 |
| Total | 100 | 100 | 100 | 100 | 100 | 100 |

TABLE 3H
Gum base formulations of mixture Exp 2 with varying amounts
of mixture Exp1, terpene resin, polyvinyl acetate, and
vegetable fats. Amounts of ingredients are shown as percentages.

| | Exp 46 | Exp 47 | Exp 48 | Exp 49 | Exp 50 | Exp 51 |
|---|---|---|---|---|---|---|
| Exp 2 | 10 | 15 | 10 | 15 | 10 | 15 |
| Terpene resin | 20 | 30 | 20 | 30 | 25 | 35 |
| Polyvinyl Acetate | 45 | 30 | 45 | 30 | 45 | 30 |
| Vegetable fats | 15 | 15 | 20 | 20 | 20 | 20 |
| Emulsifier | 4.95 | 4.95 | 4.95 | 4.95 | 4.95 | 4.95 |
| Wax | 5 | 5 | | | | |
| Antioxidant | 0.05 | 0.05 | 0.05 | 0.05 | 0.05 | 0.05 |
| Total | 100 | 100 | 100 | 100 | 100 | 100 |

TABLE 3I
Gum base formulations of mixture Exp 3 with varying amounts of
mixture Exp1, terpene resin, polyvinyl acetate, and vegetable
fats. Amounts of ingredients are shown as percentages.

| | Exp 52 | Exp 53 | Exp 54 | Exp 55 | Exp 56 | Exp 57 |
|---|---|---|---|---|---|---|
| Exp 3 | 10 | 15 | 10 | 15 | 10 | 15 |
| Terpene resin | 20 | 30 | 20 | 30 | 25 | 35 |
| Polyvinyl Acetate | 45 | 30 | 45 | 30 | 45 | 30 |
| Vegetable fats | 15 | 15 | 20 | 20 | 20 | 20 |
| Emulsifier | 4.95 | 4.95 | 4.95 | 4.95 | 4.95 | 4.95 |
| Wax | 5 | 5 | | | | |
| Antioxidant | 0.05 | 0.05 | 0.05 | 0.05 | 0.05 | 0.05 |
| Total | 100 | 100 | 100 | 100 | 100 | 100 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of producing a cultured gum base ingredient, the method comprising culturing in a culture medium a plurality of plant cells derived from a plant of a genus selected from the group consisting of Abies, Apios, Brosimum, Castilla, Cnidoscolus, Couma, Dyera, Ficus, Hevea, Isonandra, Lacmellea, Liquidambar, Manilkara, Micropholis, Palaquium, Parthenium, Picea, Pinus, Pistacia, Sideroxylon, and Taraxacum, thereby producing the cultured gum base ingredient.

2. The method of claim 1, wherein the cultured gum base ingredient comprises one or more terpenoid compounds.

3. A method of producing a cultured gum base ingredient comprising one or more terpenoid compounds, the method comprising culturing in a culture medium a plurality of plant cells derived from a plant that produces the gum base ingredient, thereby producing the cultured gum base ingredient.

4. The method of claim 3, wherein the plurality of plant cells is derived from a plant of a family selected from the group consisting of Apocynaceae, Asteraceae, Euphorbiaceae, Fabaceae, Hamamelidaceae, Moraceae, Pinaceae, Pistaciaceae, and Sapotaceae.

5. The method claim 3 or 4, wherein the plurality of plant cells is derived from a plant of a genus selected from the group consisting of Abies, Apios, Brosimum, Castilla, Cnidoscolus, Couma, Dyera, Ficus, Hevea, Isonandra, Lacmellea, Liquidambar, Manilkara, Micropholis, Palaquium, Parthenium, Picea, Pinus, Pistacia, Sideroxylon, and Taraxacum.

6. The method of any one of claims 1-5, wherein the plurality of plant cells is derived from a plant of a species selected from the group consisting of Abies amabilis, Abies balsamea, Apios americana, Brosimum utile, Castilla fallax, Cnidoscolus elasticus, Cnidoscolus tepiquensis, Couma macrocarpa, Dyera costulata, Dyera lowii, Ficus platyphylla, Hevea brasiliensis, Isonandra gutta, Lacmellea standleyi, Liquidambar orientalis, Liquidambar styraciflua, Manilkara chicle, Manilkara solimoesensis, Palaquium gutta, Palaquium leiocarpum, Parthenium argentatum, Picea rubens, Picea stichensis, Pinus ponderosa, Pinus lambertiana, Pistacia lentiscus, and Taraxacum kok-saghyz.

7. The method of any one of claims 1-5, wherein the plurality of plant cells is derived from a plant of a genus selected from the group consisting of Couma, Dyera, Hevea, Manilkara, Parthenium, Pistacia, and Taraxacum.

8. The method of any one of claims 1-5, wherein the plurality of plant cells is derived from a plant of a species selected from the group consisting of Couma macrocarpa, Dyera costulata, Dyera lowii, Hevea brasiliensis, Manilkara chicle, Manilkara solimoesensis, Parthenium argentatum, Pistacia lentiscus, and Taraxacum kok-saghyz.

9. The method of any one of claims 1-5, wherein the plurality of plant cells is derived from a plant of the genus Manilkara.

10. The method of claim 9, wherein the plurality of plant cells is derived from Manilkara chicle or Manilkara solimoesensis.

11. The method of any one of claims 2-10, wherein the one or more terpenoid compounds comprise one or more polyterpenoids, one or more triterpenoid acetates, one or more resin acids, or any combination thereof.

12. The method of any one of claims 2-11, wherein the one or more terpenoid compounds comprise luperol acetate, beta-amyrin acetate, taraxasterol acetate, alpha-amyrin acetate, polyisoprene, one or more abietic type acids, one or more pimaric type acids, or any combination thereof.

13. The method of any one of claims 1-12, wherein the cultured gum base ingredient comprises a single terpenoid compound.

14. The method of any one of claims 1-12, wherein the cultured gum base ingredient comprises a mixture of two or more terpenoid compounds.

15. The method of any one of claims 1-14, wherein the plurality of plant cells produces at least 0.01 mg/L of terpenoid compounds in the culture medium.

16. The method of any one of claims 1-14, wherein the plurality of plant cells produces at least 1 mg/L of terpenoid compounds in the culture medium.

17. The method of any one of claims 1-14, wherein the plurality of plant cells produces at least 1 g/L of terpenoid compounds in the culture medium.

18. The method of any one of claims 1-17, wherein the plurality of plant cells produces:
(i) at least 0.01 mg/L of triterpenoid acetates in the culture medium;
(ii) at least 0.01 mg/L of polyterpenoids in the culture medium; and/or
(iii) at least 0.01 mg/L of resin acids in the culture medium.

19. The method of any one of claims 1-18, wherein the plurality of plant cells produce:
(i) at least 0.01 mg/L of luperol acetate in the culture medium;
(ii) at least 0.01 mg/L of beta-amyrin acetate in the culture medium;
(iii) at least 0.01 mg/L of taraxasterol acetate in the culture medium;
(iv) at least 0.01 mg/L of alpha-amyrin acetate in the culture medium;
(v) at least 0.01 mg/L of polyisoprene in the culture medium;
(vi) at least 0.01 mg/L of one or more abietic type acids; and/or
(vii) at least 0.01 mg/L of one or more pimaric type acids.

20. The method of any one of claims 1-19, wherein the method further comprises concentrating the culture medium to produce a cultured gum base ingredient concentrate.

21. The method of any one of claims 1-20, wherein the method further comprises separating the cultured gum base ingredient from the culture medium or gum base ingredient concentrate to produce an isolated or purified cultured gum base ingredient.

22. A cultured gum base ingredient produced according to the method of any one of claims 1-21.

23. A cultured gum base ingredient concentrate produced according to the method of claim 20, comprising terpenoid compounds in an amount of at least 5% by weight.

24. An isolated or purified cultured gum base ingredient produced according to the method of claim 21, comprising terpenoid compounds in an amount of at least 5% by weight.

25. The cultured gum base ingredient concentrate of claim 23, or the isolated or purified cultured gum base ingredient of claim 24, comprising terpenoid compounds in an amount of at least 25% by weight.

26. The cultured gum base ingredient concentrate of claim 23, or the isolated or purified cultured gum base ingredient of claim 24, comprising terpenoid compounds in an amount of at least 50% by weight.

27. The cultured gum base ingredient concentrate of any one of claims 23, 25, and 26, or the isolated or purified cultured gum base ingredient of any one of claims 24-26, comprising:
(i) triterpenoid acetates in an amount of at least 5% by weight;
(ii) polyterpenoids in an amount of at least 5% by weight; and/or
(iii) resin acids in an amount of at least 5% by weight.

28. The cultured gum base ingredient concentrate of any one of claims 23 and 25-27, or the isolated or purified cultured gum base ingredient of claim 24-27, comprising:
(i) luperol acetate in an amount of at least 1% by weight;
(ii) beta-amyrin acetate in an amount of at least 1% by weight;
(iii) taraxasterol acetate in an amount of at least 1% by weight;
(iv) alpha-amyrin acetate in an amount of at least 1% by weight;
(v) polyisoprene in an amount of at least 1% by weight;
(vi) at least 0.01 mg/L of one or more abietic type acids; and/or
(vii) at least 0.01 mg/L of one or more pimaric type acids.

29. A process for producing a food product, the process comprising a step of including the cultured gum base ingredient of claim 22, the cultured gum base ingredient concentrate of any one of claims 23 and 25-26, or the isolated or purified gum base ingredient of any one of claims 24-26 in the food product.

30. A food product produced according to the method of claim 29.

31. A gum base produced according to the method of claim 29.

32. The gum base of claim 31, wherein the gum base has a complex viscosity of between 10⁴ and 10⁸ Pa*s at a temperature of between 20° C. and 25° C., and between 10² and 10⁵ Pa*s at a temperature of 60° C. and above, as measured by linear viscoelastic (LVE) rheology.

33. The gum base of claim 31 or 32, wherein the gum base comprises the cultured gum base ingredient in an amount of between about 1% and about 25% by weight of the gum base.

34. The gum base of claim 31 or 32, wherein the gum base comprises the cultured gum base ingredient in an amount of between about 5% and about 20% by weight of the gum base.

35. The gum base of any one of claims 31-34, further comprising one or more additional components selected from the group consisting of filler component, terpene resin, gum resin, polyvinyl acetate, one or more vegetable fats, one or more emulsifiers, one or more waxes, one or more antioxidants, or any combination thereof.

36. The gum base of claim 35, wherein the gum base comprises:
(i) filler component in an amount of between about 0.01% and about 30% by weight of the gum base;
(ii) terpene resin in an amount of between about 5% and about 50% by weight of the gum base;
(iii) polyvinyl acetate in an amount of between about 0.01% and about 60% by weight of the gum base;
(iv) one or more vegetable fats in an amount of between about 5% and about 30% by weight of the gum base;
(v) one or more emulsifiers in an amount of between about 0.01% and about 10% by weight of the gum base;
(vi) one or more waxes in an amount of between about 0.01% and about 15% by weight of the gum base; and/or
(vii) one or more antioxidants in an amount of between about 0.01% and about 1% by weight of the gum base.

37. The gum base of claim 36, wherein the filler component is talc, calcium carbonate, or a combination thereof.

38. A chewing gum comprising the gum base of any one of claims 31-37.

39. The chewing gum of claim 38, wherein the chewing gum comprises the gum base in an amount of between 5% and 95% by weight of the chewing gum.

40. The chewing gum of claim 38, wherein the chewing gum comprises the gum base in an amount of between 20% and 40% by weight of the chewing gum.

41. The chewing gum of any one of claims 38-40, wherein all of the components of the chewing gum are derived from natural sources.

42. The chewing gum of any one of claims 38-41, wherein all of the components of the chewing gum are derived from plant sources.

43. The chewing gum of any one of claims 38-42, wherein the chewing gum is free of artificial and synthetic ingredients.

44. The chewing gum of any one of claims 38-43, wherein the chewing gum is free of ingredients that are derived from petroleum.
